(19) United States
(12) Patent Application Publication
(10) Pub. No.: US 2025/0205309 A1
(43) Pub. Date: Jun. 26, 2025

(54) COMBINATIONS OF IL15/IL15R ALPHA HETERODIMERIC FC-FUSION PROTEINS AND FCRH5XCD3 BISPECIFIC ANTIBODIES FOR THE TREATMENT OF BLOOD CANCERS

(71) Applicants: Genentech, Inc., South San Francisco, CA (US); Xencor, Inc., Pasadena, CA (US)

(72) Inventors: Alexander Joachim Paul Ungewickell, South San Francisco, CA (US); James Niall Cooper, South San Francisco, CA (US); Danielle Shin, South San Francisco, CA (US); Teemu Tapani Junttila, South San Francisco, CA (US); Patrick Gwynn Holder, South San Francisco, CA (US); Ji Li, South San Francisco, CA (US)

(21) Appl. No.: 19/010,767

(22) Filed: Jan. 6, 2025

Related U.S. Application Data
(63) Continuation of application No. PCT/US2023/069713, filed on Jul. 6, 2023.
(60) Provisional application No. 63/367,900, filed on Jul. 7, 2022.
(60) Provisional application No. 63/504,524, filed on May 26, 2023.

(51) Int. Cl. A61K 38/20; A61K 38/17; A61K 39/00; A61P 35/00; C07K 16/24; C07K 16/28

(52) U.S. Cl. CPC A61K 38/2086; A61K 38/1793; A61P 35/00; C07K 16/248; C07K 16/2809; C07K 16/283; A61K 2039/54; A61K 2039/545; C07K 2317/31; C07K 2317/565

(57) ABSTRACT

The disclosure provides methods of treating a blood cancer, such as multiple myeloma, by administering a combination of a heterodimeric protein comprising a first monomer comprising an IL15 protein-Fc domain fusion and a second monomer comprising an IL15Rα protein-Fc domain fusion, such as XmAb24306, and a FcRH5xCD3 bispecific antibody, such as cevostamab.

### CROSS-REFERENCE TO RELATED APPLICATIONS

This application claims priority to and benefit of U.S. Provisional Application No. 63/367,900, filed Jul. 7, 2022, and U.S. Provisional Application No. 63/504,524, filed May 26, 2023, the contents of each application are hereby incorporated by reference in their entireties.

### TECHNICAL FIELD

The present disclosure pertains to the field of treatment of blood cancer, such as multiple myeloma, using a combination of an IL15-IL15Rα heterodimeric Fc-fusion protein and an FcRH5xCD3 bispecific antibody.

### SEQUENCE LISTING

The instant application contains a Sequence Listing which has been submitted electronically in XML format and is hereby incorporated by reference in its entirety. Said XML copy, created on Jul. 6, 2023, is named 000218-0060-WO1 SL.xml and is 31,425 bytes in size.

### BACKGROUND

Most blood cancers (or hematologic cancers) start in the bone marrow and result from abnormal blood cells growing out of control, interrupting the function of normal blood cells, which fight off infection and produce new blood cells. Multiple myeloma (MM), one type of blood cancer, is an incurable neoplasm characterized by the proliferation and accumulation of malignant plasma cells in the bone marrow that leads to the overproduction of monoclonal proteins (M-proteins) detectable in blood or urine of most subjects. A diagnosis of MM affects approximately 30,000 people every year in the United States (Siegel et a al. 2019), and approximately 160,000 people are diagnosed with MM annually worldwide (Bray et al. 2018). End-organ damage resulting from MM includes hypercalcemia, renal insufficiency, anemia, and lytic bone lesions. MM remains incurable despite advances in treatment, with an estimated median survival of 8-10 years for standard-risk and 2-3 years from high-risk myeloma, even with aggressive treatments such as autologous stem cell transplantation (ASCT) (Mikhael et al. 2013). Increased survival has been achieved with the introduction of proteasome inhibitors (PIS) such as bortezomib (Velcade® U.S. Package Insert [USPI]), immunomodulatory drugs (IMiDs) such as lenalidomide (Revlimid® USPI), and monoclonal antibodies such as daratumumab (Darzalex® USPI, Darzalex-Faspro™ USPI). Other agents with novel mechanisms of action that have received U.S. Food and Drug Administration approval for the treatment of MM include the nuclear export inhibitor Selinexor (Xpovio™ USPI) and belantamab mafodotin-bmlf (Blenrep USPI).

Despite the significant progress in treatment options, most MM patients eventually relapse. Relapsed/refractory multiple myeloma (R/R MM) continues to constitute a significant unmet medical need, with median overall survival of less than one year in subjects who have disease refractory to anti-CD38 monoclonal antibodies (Chari et al. 2019; Ghandi et al. 2019). Several approaches that direct the human immune system to target and destroy malignant plasma cells are currently being explored in clinical settings, including T cell-engaging bispecific antibodies and chimeric antigen receptor [CAR] T cells. Emerging data from clinical studies using these agents suggest that manipulation of the subject's immune system is a potentially promising approach for the treatment of R/R MM (Moreau et al. 2019; Caraccio et al. 2020).

### SUMMARY

In a first aspect, the present disclosure provides a method of treating a blood cancer in a subject in need thereof, the method comprising administering to the subject a therapeutically effective amount of (a) an IL15-IL15Rα heterodimeric Fc-fusion protein and (b) an FcRH5xCD3 bispecific antibody or a fragment thereof that binds both antigens.

In a second aspect, the present disclosure provides use of a therapeutically effective amount of (a) an IL15-IL15Rα heterodimeric Fc-fusion protein and (b) an FcRH5xCD3 bispecific antibody or a fragment thereof that binds both antigens in the manufacture of one or more medicaments for the treatment of a blood cancer in a subject in need thereof.

In a third aspect, the present disclosure provides a therapeutically effective amount of (a) an IL15-IL15Rα heterodimeric Fc-fusion protein and (b) an FcRH5xCD3 bispecific antibody or a fragment thereof that binds both antigen for use in treating a blood cancer in a subject in need thereof.

In some embodiments, the IL15-IL15Rα heterodimeric Fc-fusion protein comprises: (a) a first fusion protein comprising an interleukin-15 (IL-15) protein covalently attached to the N-terminus of a first Fc domain via a first domain linker, wherein the IL-15 protein comprises the amino acid sequence of SEQ ID NO: 5 and wherein the first Fc domain is a variant of a human IgG1 Fc domain; and (b) a second fusion protein comprising an IL-15 receptor alpha (IL-15Rα) protein fragment covalently attached to the N-terminus of a second Fc domain via a second domain linker, wherein the IL-15Rα protein comprises the amino acid sequence of SEQ ID NO: 4 and wherein the second Fc domain is a variant of a human IgG1 Fc domain.

In some embodiments, the IL15-IL15Rα heterodimeric Fc-fusion protein comprises: (a) a first fusion protein comprising an interleukin-15 (IL-15) protein covalently attached to the N-terminus of a first Fc domain via a first domain linker, wherein the IL-15 protein comprises the amino acid sequence of SEQ ID NO: 5 and wherein the first Fc domain comprises the amino acid sequence of SEQ ID NO: 6; and (b) a second fusion protein comprising an IL-15 receptor alpha (IL-15Rα) protein fragment covalently attached to the N-terminus of a second Fc domain via a second domain linker, wherein the IL-15Rα protein comprises the amino acid sequence of SEQ ID NO: 4 and wherein the second Fc domain comprises the amino acid sequence of SEQ ID NO: 7.

In some embodiments, the first domain linker comprises the amino acid sequence of SEQ ID NO: 8. In some embodiments, the second domain linker comprises the amino acid sequence of SEQ ID NO: 8. In some embodiments, the first domain linker comprises the amino acid sequence of SEQ ID NO: 8 and the second domain linker comprises the amino acid sequence of SEQ ID NO: 8.

In some embodiments, the first fusion protein comprises the amino acid sequence of SEQ ID NO: 9. In some embodiments, the second fusion protein comprises the amino acid sequence of SEQ ID NO: 10. In some embodiments, the heterodimeric protein comprises a first monomer comprising the amino acid sequence set forth in SEQ ID NO: 9, and a second monomer comprising the amino acid sequence set forth in SEQ ID NO: 10.

In some embodiments, the IL15-IL15Rα heterodimeric Fc-fusion protein is XmAb24306.

In some embodiments, the FcRH5xCD3 bispecific antibody or a fragment thereof that binds both antigens comprises an anti-FcRH5 light chain variable region, an anti-FcRH5 heavy chain variable region, an anti-CD3 light chain variable region, and an anti-CD3 heavy chain variable region.

In some embodiments, the anti-FcRH5 light chain variable region comprises a light chain complementarity determining region-1 (CDR-L1) comprising the amino acid sequence of SEQ ID NO: 11, a CDR-L2 comprising the amino acid sequence of SEQ ID NO: 12, and a CDR-L3 comprising the amino acid sequence of SEQ ID NO: 13; and the anti-FcRH5 heavy chain variable region comprises a heavy chain complementarity determining region-1 (CDR-H1) comprising the amino acid sequence of SEQ ID NO: 14, a CDR-H2 comprising the amino acid sequence of SEQ ID NO: 15, and a CDR-H3 comprising the amino acid sequence of SEQ ID NO: 16.

In some embodiments, the anti-FcRH5 light chain variable region comprises the amino acid sequence of SEQ ID NO: 17. In some embodiments, the anti-FcRH5 heavy chain variable region comprises the amino acid sequence of SEQ ID NO: 18. In some embodiments, the anti-FcRH5 light chain comprises the amino acid sequence of SEQ ID NO: 19. In some embodiments, the anti-FcRH5 heavy chain comprises the amino acid sequence of SEQ ID NO: 20.

In some embodiments, the anti-CD3 light chain variable region comprises a light chain complementarity determining region-1 (CDR-L1) comprising the amino acid sequence of SEQ ID NO: 21, a CDR-L2 comprising the amino acid sequence of SEQ ID NO: 22, and a CDR-L3 comprising the amino acid sequence of SEQ ID NO: 23; and the anti-CD3 heavy chain variable region comprises a heavy chain complementarity determining region-1 (CDR-H1) comprising the amino acid sequence of SEQ ID NO: 24, a CDR-H2 comprising the amino acid sequence of SEQ ID NO: 25, and a CDR-H3 comprising the amino acid sequence of SEQ ID NO: 26.

In some embodiments, the anti-CD3 light chain variable region comprises the amino acid sequence of SEQ ID NO: 27. In some embodiments, the anti-CD3 heavy chain variable region comprises the amino acid sequence of SEQ ID NO: 28. In some embodiments, the anti-CD3 light chain comprises the amino acid sequence of SEQ ID NO: 29. In some embodiments, the anti-CD3 heavy chain comprises the amino acid sequence of SEQ ID NO: 30.

In some embodiments, the anti-FcRH5 light chain variable region comprises the amino acid sequence of SEQ ID NO: 17; the anti-FcRH5 heavy chain variable region comprises the amino acid sequence of SEQ ID NO: 18; the anti-CD3 light chain variable region comprises the amino acid sequence of SEQ ID NO: 27; and the anti-CD3 heavy chain variable region comprises the amino acid sequence of SEQ ID NO: 28.

In some embodiments, the anti-FcRH5 light chain comprises the amino acid sequence of SEQ ID NO: 19; the anti-FcRH5 heavy chain comprises the amino acid sequence of SEQ ID NO: 20; the anti-CD3 light chain comprises the amino acid sequence of SEQ ID NO: 29; and the anti-CD3 heavy chain comprises the amino acid sequence of SEQ ID NO: 30.

In some embodiments, the FcRH5xCD3 bispecific antibody is cevostamab.

In some embodiments, the blood cancer of this disclosure is selected from the group consisting of leukemia, acute myeloid leukemia, adult acute lymphoblastic leukemia, chronic lymphocytic leukemia, lymphoma, non-Hodgkin's lymphoma, B-cell non-Hodgkin's lymphoma, and multiple myeloma. In some embodiments, the blood cancer is multiple myeloma. In some embodiments, the multiple myeloma is relapsed or refractory multiple myeloma. In some embodiments, the blood cancer is B-cell non-Hodgkin's lymphoma. In some embodiments, the blood cancer is chronic lymphocytic leukemia.

In some embodiments, the subject has been previously administered one or more prior treatments.

In some embodiments, the prior treatment of this disclosure is an immunomodulatory drug, a proteasome inhibitor, or an anti-CD38 monoclonal antibody. In some embodiments, the immunomodulatory drug is selected from the group consisting of lenalidomide, thalidomide, and pomalidomide. In some embodiments, the proteasome inhibitor is selected from the group consisting of bortezomib, carfilzomib, and ixazomib. In some embodiments, the anti-CD38 monoclonal antibody is selected from the group consisting of daratumumab, isatuximab, mezagitamab, and felzartamab.

In some embodiments, the IL15-IL15Rα heterodimeric Fc-fusion protein (e.g., XmAb24306) is administered at a dose selected from the group consisting of about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.1 mg/kg, about 0.12 mg/kg, about 0.16 mg/kg, about 0.2 mg/kg, about 0.24 mg/kg and about 0.32 mg/kg body weight. In some embodiments, the IL15-IL15Rα heterodimeric Fc-fusion protein (e.g., XmAb24306) is administered at a dose selected from the group consisting of about 0.01 mg/kg, about 0.02 mg/kg, about 0.04 mg/kg, about 0.06 mg/kg, about 0.09 mg/kg and about 0.12 mg/kg body weight.

In some embodiments, the IL15-IL15Rα heterodimeric Fc-fusion protein (e.g., XmAb24306) is administered at a dose selected from the group consisting of 0.0025 mg/kg, 0.005 mg/kg, 0.01 mg/kg, 0.015 mg/kg, 0.02 mg/kg, 0.025 mg/kg, 0.03 mg/kg, 0.04 mg/kg, 0.05 mg/kg, 0.06 mg/kg, 0.08 mg/kg, 0.10 mg/kg, 0.16 mg/kg, 0.20 mg/kg, 0.24 mg/kg and 0.32 mg/kg body weight. In some embodiments, the IL15-IL15Rα heterodimeric Fc-fusion protein (e.g., XmAb24306) is administered at a dose selected from the group consisting of 0.01 mg/kg, 0.02 mg/kg, 0.04 mg/kg, 0.06 mg/kg, 0.09 mg/kg, and 0.12 mg/kg body weight.

In some embodiments, the IL15-IL15Rα heterodimeric Fc-fusion protein (e.g., XmAb24306) is administered at a frequency selected from the group consisting of Q1W, Q2W, Q3W, Q4W, Q5W and Q6W. In some embodiments, the IL15-IL15Rα heterodimeric Fc-fusion protein (e.g., XmAb24306) is administered at a frequency of Q1W in one or more cycles. In some embodiments, the IL15-IL15Rα heterodimeric Fc-fusion protein (e.g., XmAb24306) is administered at a frequency of Q2W in one or more cycles. In some embodiments, the IL15-IL15Rα heterodimeric Fc-fusion protein (e.g., XmAb24306) is administered at a frequency of Q4W in one or more cycles.

In some embodiments, the IL15-IL15Rα heterodimeric Fc-fusion protein (e.g., XmAb24306) is administered intravenously.

In some embodiments, the IL15-IL15Rα heterodimeric Fc-fusion protein (e.g., XmAb24306) and said FcRH5xCD3 bispecific antibody (e.g., cevostamab) or fragment thereof that binds both antigens are administered simultaneously. In some embodiments, the IL15-IL15Rα heterodimeric Fc-fusion protein (e.g., XmAb24306) and said FcRH5xCD3 bispecific antibody (e.g., cevostamab) or fragment thereof that binds both antigens are administered sequentially.

In some embodiments, the FcRH5xCD3 bispecific antibody (e.g., cevostamab) or fragment thereof that binds both antigens is administered at a frequency selected from the group consisting of Q1W, Q2W, Q3W, Q4W, Q5W and Q6W in one or more cycles. In some embodiments, the FcRH5xCD3 bispecific antibody (e.g., cevostamab) or fragment thereof that binds both antigens is administered at a frequency of Q2W in one or more cycles. In some embodiments, the IL15-IL15Rα heterodimeric Fc-fusion protein (e.g., XmAb24306) is administered at a frequency of Q4W, and wherein said FcRH5xCD3 bispecific antibody (e.g., cevostamab) or fragment thereof that binds both antigens is administered at a frequency of Q2W in one or more cycles.

In some embodiments, each of the one or more cycles is a four-week cycle. In some embodiments, the FcRH5xCD3 bispecific antibody (e.g., cevostamab) or a fragment thereof that binds both antigens is administered Q2W for six four-week cycles and administered Q4W in the seventh and any subsequent four-week cycle. In some embodiments, the FcRH5xCD3 bispecific antibody (e.g., cevostamab) or a fragment thereof that binds both antigens is administered on Days 1 and 15 for six four-week cycles and administered on Day 1 in the seventh and any subsequent four-week cycle.

In some embodiments, the FcRH5xCD3 bispecific antibody (e.g., cevostamab) is administered at a dose of about 132 mg to about 198 mg. In some embodiments, the FcRH5xCD3 bispecific antibody (e.g., cevostamab) is administered at a dose of about 132 mg. In some embodiments, the FcRH5xCD3 bispecific antibody (e.g., cevostamab) is administered at a dose of about 132 mg at a frequency of Q2W in one or more 28-day cycles. In some embodiments, the FcRH5xCD3 bispecific antibody (e.g., cevostamab) is administered at a dose of about 160 mg. In some embodiments, the FcRH5xCD3 bispecific antibody (e.g., cevostamab) is administered at a dose of about 198 mg.

In some embodiments, the one or more priming doses of the FcRH5xCD3 bispecific antibody (e.g., cevostamab) or a fragment thereof that binds both antigens is administered to the subject during a pre-phase before the first treatment cycle. In some embodiments, the pre-phase is seven days. In some embodiments, the priming dose is about 3.6 mg of the FcRH5xCD3 bispecific antibody (e.g., cevostamab). In some embodiments, the two priming doses of the FcRH5xCD3 bispecific antibody (e.g., cevostamab) or a fragment thereof that binds both antigens are administered to the subject. In some embodiments, about 3.6 mg of the FcRH5xCD3 bispecific antibody (e.g., cevostamab) is administered between the two priming doses (i.e., the amount administered in the first priming dose and the amount administered in the second priming dose add up to about 3.6 mg). In some embodiments, the first priming dose is administered on pre-phase Day 1. In some embodiments, the second priming dose is administered between pre-phase Days 2-4. In some embodiments, the minimum interval between the end of the first priming dose and the initiation of the second priming dose is 20 hours. In some embodiments, the FcRH5xCD3 bispecific antibody (e.g., cevostamab) or a fragment thereof that binds both antigens is administered between the two priming doses. In some embodiments, the first priming dose is about 0.3 mg and the second priming dose is about 3.3 mg.

In some embodiments, the subject is administered a first priming dose of about 0.3 mg of the FcRH5xCD3 bispecific antibody (e.g., cevostamab) on pre-phase Day 1, a second priming dose of about 3.3 mg of the FcRH5xCD3 bispecific antibody (e.g., cevostamab) between pre-phase Days 2-4 and, thereafter, at a dose of about 132 mg of the FcRH5xCD3 bispecific antibody (e.g., cevostamab) on Days 1 and 15 for six four-week cycles and on Day 1 for the seventh and any subsequent four-week cycles.

In some embodiments, the FcRH5xCD3 bispecific antibody (e.g., cevostamab) is administered intravenously.

In some embodiments, the method further comprises administering to the subject a therapeutically effective amount of tocilizumab. In some embodiments, the subject has suffered a cytokine release syndrome (CRS) event. In some embodiments, the tocilizumab is administered to subjects that remain refractory to a corticosteroid 24 hours after the first corticosteroid dose. In some embodiments, the tocilizumab is administered at a dose of 8 mg/kg. In some embodiments, the tocilizumab is administered at a dose of 8 mg/kg if the subject's weight is ≥30 kg. In some embodiments, the tocilizumab is administered at a dose of 12 mg/kg. In some embodiments, the tocilizumab is administered at a dose of 12 mg/kg if the patient's weight is <30 kg.

In some embodiments, the tocilizumab is administered intravenously. In some embodiments, the tocilizumab is administered every 8 hours.

A fourth aspect of the present disclosure provides a method of treating multiple myeloma in a subject in need thereof, the method comprising administering to the subject a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30, wherein XmAb24306 is administered intravenously at a dose from about 0.02 mg/kg to about 0.06 mg/kg and cevostamab is administered intravenously at a dose of about 132 mg.

A fifth aspect of the present disclosure provides use of a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30 in the manufacture of one or more medicaments for treating multiple myeloma in a subject in need thereof, wherein XmAb24306 is formulated to be administered intravenously at a dose from about 0.02 mg/kg to about 0.06 mg/kg and cevostamab is formulated to be administered intravenously at a dose of about 132 mg.

A sixth aspect of the present disclosure provides a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30 for use in treating multiple myeloma in a subject in need thereof, wherein XmAb24306 is administered intravenously at a dose from about 0.02 mg/kg to about 0.06 mg/kg and cevostamab is administered intravenously at a dose of about 132 mg.

In some embodiments, the treatment further comprises administering to the subject one or more priming doses of cevostamab at a total dose of about 3.6 mg during a seven-day pre-phase before the first treatment cycle, wherein the priming dose is administered as a single dose. In some embodiments, the treatment further comprises administering to the subject one or more priming doses of cevostamab at a total dose of about 3.6 mg during a seven-day pre-phase before the first treatment cycle, wherein the priming dose is administered as two doses. In some embodiments, the first priming dose is about 0.3 mg and the second priming dose is about 3.3 mg. In some embodiments, the first priming dose is administered on Day 1 of the pre-phase and wherein the second priming dose is administered on Day 2, 3 or 4 of the pre-phase.

In some embodiments, XmAb24306 is administered at a frequency of Q4W for one or more cycles.

In some embodiments, cevostamab is administered at a frequency of Q2W for one or more cycles. In some embodiments, cevostamab is administered at a frequency of Q4W for one or more cycles.

In some embodiments, XmAb24306 is administered intravenously at a frequency of Q4W for at least seven four-week cycles, and wherein a first priming dose of 0.3 mg of cevostamab is administered intravenously on pre-phase Day 1, a second priming dose of 3.3 mg of cevostamab is administered intravenously between pre-phase Days 2-4, wherein the pre-phase is seven days, and, thereafter, at a dose of 132 mg of cevostamab is administered intravenously on Days 1 and 15 for six four-week cycles and on Day 1 for the seventh and any subsequent four-week cycles.

A seventh aspect of the present disclosure provides a method of treating multiple myeloma in a subject in need thereof, the method comprising administering to the subject a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30, wherein XmAb24306 is administered intravenously at a dose from about 0.02 mg/kg to about 0.06 mg/kg at a frequency of Q4W for at least seven four-week cycles, and wherein a first priming dose of 0.3 mg of cevostamab is administered intravenously on pre-phase Day 1, a second priming dose of 3.3 mg of cevostamab is administered intravenously between pre-phase Days 2-4, wherein the pre-phase is seven days, and, thereafter, at a dose of 132 mg of cevostamab is administered intravenously on Days 1 and 15 for six four-week cycles and on Day 1 for the seventh and any subsequent four-week cycles.

An eighth aspect of the present disclosure provides use of a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30 in the manufacture of one or more medicaments for treating multiple myeloma in a subject in need thereof, wherein XmAb24306 is formulated to be administered intravenously at a dose from about 0.02 mg/kg to about 0.06 mg/kg at a frequency of Q4W for at least seven four-week cycles, wherein cevostamab is formulated to be administered intravenously at a dose of 132 mg on Days 1 and 15 for six four-week cycles and on Day 1 for the seventh and any subsequent four-week cycles; and wherein the treatment further comprises a seven-day pre-phase in which a first priming dose of 0.3 mg of cevostamab is administered intravenously on pre-phase Day 1, and a second priming dose of 3.3 mg of cevostamab is administered intravenously between pre-phase Days 2-4.

A ninth aspect of the present disclosure provides a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30 for use in treating multiple myeloma in a subject in need thereof, wherein XmAb24306 is administered intravenously at a dose from about 0.02 mg/kg to about 0.06 mg/kg at a frequency of Q4W for at least seven four-week cycles, and wherein a first priming dose of 0.3 mg of cevostamab is administered intravenously on pre-phase Day 1, a second priming dose of 3.3 mg of cevostamab is administered intravenously between pre-phase Days 2-4, wherein the pre-phase is seven days, and, thereafter, at a dose of 132 mg of cevostamab is administered intravenously on Days 1 and 15 for six four-week cycles and on Day 1 for the seventh and any subsequent four-week cycles.

### BRIEF DESCRIPTION OF THE DRAWINGS

FIGS. 1A-1B provide graphs measuring in vitro pharmacologic activity of cevostamab in combination with XmAb24306 in primary bone marrow mononuclear cells (BMMC) from multiple myeloma (MM) subjects (i.e., donors 1-3). FIG. 1A provides a graph showing percentage change of CD8⁺ T cell number compared to untreated samples in BMMC in MM subjects. FIG. 1B provides a graph showing the percentage of MM tumor cell (CD38^{high}, CD138⁺) lysis. MM=multiple myeloma. Data are shown as means±SD of the replicates. Paired t test was used for statistical analysis.

FIGS. 2A-2C provide graphs showing the levels of CD69 (FIG. 2A), CD25 (FIG. 2B) and CD122 (IL-2R/IL-15Rb; FIG. 2C) expression in human CD4⁺ T cells, CD8⁻ T cells, and natural killer (NK) cells following cevostamab treatment in vitro. Human PBMCs from three healthy donors were co-cultured with a multiple myeloma cell line (MOLP-2 cells) in the presence of 5000 pM of cevostamab or isotype control or in the absence of treatment for 30 minutes, 2 hours, 4 hours, 24 hours and 48 hours. The mean fluorescence intensity level for CD69 (FIG. 2A), CD25 (FIG. 2B) and CD122 (FIG. 2C) was measured on CD4⁺ T cells, CD8⁺ T cells and on NK cells. Cevostamab induced upregulation of CD69, CD25, and CD122 in CD4⁺ T cells and CD8⁺ T cells. Data are shown as means±SD of the replicates. MFI=mean fluorescence intensity; NK=natural killer cells; TDB=T-cell-dependent bispecific antibody.

FIG. 3 provides the amino acid sequences for XmAb24306 monomer 1 (SEQ ID NO: 9) and XmAb24306 monomer 2 (SEQ ID NO: 10). In the monomer 1 sequences, the IL15 portion is underlined, the linker is offset with slashes and is bolded and underlined, and the Fc portion follows the second slash and does not contain any formatting. In the monomer 2 sequences, the IL15Rα portion is underlined, the linker is offset with slashes and is bold and underlined, and the Fc portion follows the second slash and does not contain any formatting.

FIGS. 4A and 4B provides the amino acid sequences for the human IL-15 precursor protein (full-length human IL-15) (SEQ ID NO: 2), the mature or truncated human IL-15 protein (SEQ ID NO: 1), the full-length human IL-15Rα protein (SEQ ID NO: 3), the extracellular domain of the human IL-15Rα protein (SEQ ID NO: 31), and the sushi domain of the human IL-15Rα protein (SEQ ID NO: 4).

FIG. 5 provides a representative treatment schedule for a combination therapy comprising XmAb24306 and cevostamab. DLT=dose-limiting toxicity.

FIG. 6 is the combination study schema for an IL15/IL15Rα heterodimeric protein (e.g., XmAb24306) and cevostamab, showing subjects enrolled in two stages: a dose-escalation stage and an expansion stage and details on these two stages. XmAb=IL15/IL15Rα heterodimeric protein (e.g., XmAb24306); cevos=cevostamab; PK=pharmacokinetic; PD=pharmacodynamic.

FIG. 7 provides a representative treatment schedule for cevostamab monotherapy. DLT=dose-limiting toxicity.

### DETAILED DESCRIPTION

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature.

The term “herein” means the entire application.

It should be understood that any of the embodiments described herein, including those described under different aspects of the disclosure and different parts of the specification (including embodiments described only in the Examples) can be combined with one or more other embodiments disclosed herein, unless explicitly disclaimed or improper. Combination of embodiments are not limited to those specific combinations claimed via the multiple dependent claims.

Any publications, patents and published patent applications referred to in this application are specifically incorporated by reference herein. In case of conflict, the present specification, including its specific definitions, will control.

Throughout this specification, the word “comprise” or variations such as “comprises” or “comprising,” which is synonymous with “including,” “containing,” or “characterized by,” is inclusive or open-ended and does not exclude additional, unrecited elements or method steps.

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “consisting of” excludes any element, step, or ingredient not specifically recited.

The term “consisting essentially of” limits the scope of a disclosure to the specified materials or steps and those that do not materially affect the basic and novel characteristic(s) of the disclosure.

Any example(s) following the term “e.g.” or “for example” is not meant to be exhaustive or limiting.

The articles “a,” “an” and “the” are used herein to refer to one or to more than one (i.e., to at least one) of the grammatical object of the article. By way of example, “an element” means one element or more than one element.

As used herein, the term “about” modifying the quantity of an ingredient, parameter, calculation, or measurement in the compositions employed in the methods of the disclosure refers to the variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be typically within 10%, more typically still within 5%, of a given value or range. The term “about” also encompasses amounts that differ due to different equilibrium conditions for a composition resulting from a particular initial mixture. Whether or not modified by the term “about,” the paragraphs include equivalents to the quantities. Reference to “about” a value or parameter herein includes (and describes) embodiments that are directed to that value or parameter per se. For example, description referring to “about X” includes description of “X.” Numeric ranges are inclusive of the numbers defining the range.

The term “or” as used herein should be understood to mean “and/or,” unless the context clearly indicates otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The disclosure of a range should also be considered as disclosure of the endpoints of that range.

Exemplary methods and materials are described herein, although methods and materials similar or equivalent to those described herein can also be used in the practice or testing of the present application. The materials, methods, and examples are illustrative only and not intended to be limiting.

### Definitions

The following terms, unless otherwise indicated, shall be understood to have the following meanings:

The term “ablation,” as used herein, refers to a decrease or removal of activity. Thus, for example, “ablating FcγR binding” means that the Fc region amino acid variant has less than 50% starting binding as compared to an Fc region not containing the specific variant, with less than 70%, less than 80%, less than 90%, less than 95% or less than 98% loss of activity being preferred, and in general, with the activity being below the level of detectable binding in a BIACORE® assay (Pharmacia Biosensor AB, Uppsala, Sweden and Piscataway, N.J.). Unless otherwise noted, the Fc domains described herein retain binding to the FcRn receptor.

“Administering” or “administration of” a substance, a compound or an agent to a subject refers to the contact of that substance, compound or agent to the subject or a cell, tissue, organ or bodily fluid of the subject. For example, a compound or an agent can be administered intravenously or subcutaneously. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a subject to self-administer a drug, or to have the drug administered by another and/or who provides a subject with a prescription for a drug is administering the drug to the subject.

As used herein, the term “affinity” of a molecule refers to the strength of interaction between the molecule and a binding partner, such as a receptor, a ligand or an antigen. A molecule's affinity for its binding partner is typically expressed as the binding affinity equilibrium dissociation constant (KD) of a particular interaction, wherein the lower the KD, the higher the affinity. A KD binding affinity constant can be measured by surface plasmon resonance, for example using the BIACORE® system (Pharmacia Biosensor AB, Uppsala, Sweden and Piscataway, N.J.) See also, Jonsson et al., Ann. Biol. Clin. 51:19 26 (1993); Jonsson et al., Biotechniques 11:620 627 (1991); Jonsson et al., J. Mol. Recognit. 8:125 131 (1995); Johnsson et al., Anal. Biochem. 198:268 277 (1991); Hearty S et al., Methods Mol Biol. 907:411-42 (2012), each incorporated herein by reference. The KD may also be measured using a KinExA® system (Sapidyne Instruments, Hanover, Germany and Boise, ID). In some embodiments, the IL-15 variant of the heterodimeric protein described herein has reduced binding affinity towards IL-2/IL-15By receptor, compared with wild-type IL-15. In some embodiments, the first and/or the second Fc variant of the heterodimeric protein described herein has reduced affinity towards human, cynomolgus monkey, and mouse Fcγ receptors. In some embodiments, the first and/or the second Fc variant of the heterodimeric protein described herein does not bind to human, cynomolgus monkey, and mouse Fcγ receptors.

The terms “amino acid” and “amino acid identity,” as used herein, refer to one of the 20 naturally occurring amino acids that are coded for by DNA and RNA.

The term “amino acid substitution” or “substitution,” as used herein, refers to the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not considered an amino acid substitution.

The terms “amino acid insertion,” “amino acid addition” or “addition” or “insertion,” as used herein, refer to the addition of an amino acid sequence at a particular position in a parent polypeptide sequence. For example, -233E, _233E or 233E designates an insertion of glutamic acid after position 233 and before position 234. Additionally, -233ADE, _233ADE or 233ADE designates an insertion of AlaAspGlu after position 233 and before position 234.

The term “amino acid deletion” or “deletion,” as used herein, refers to the removal of an amino acid sequence at a particular position in a parent polypeptide sequence. For example, E233- or E233#, E233( ), E233_or E233del designates a deletion of glutamic acid at position 233. Additionally, EDA233-, EDA233_ or EDA233# designates a deletion of the sequence GluAspAla that begins at position 233.

As used herein, the term “antibody” or “Ab” refers to an immunoglobulin molecule (e.g., complete antibodies, antibody fragment or modified antibodies) capable of recognizing and binding to a specific target or antigen, such as a carbohydrate, polynucleotide, lipid, polypeptide, etc., through at least one antigen recognition site, located in the variable region of the immunoglobulin molecule. As used herein, the term “antibody” can encompass any type of antibody, including but not limited to monoclonal antibodies, polyclonal antibodies, human antibodies, engineered antibodies (including humanized antibodies, fully human antibodies, chimeric antibodies, single-chain antibodies, artificially selected antibodies, CDR-granted antibodies, etc.) that specifically bind to a given antigen. In some embodiments, “antibody” and/or “immunoglobulin” (Ig) refers to a polypeptide comprising at least two heavy (H) chains (about 50-70 kDa) and two light (L) chains (about 25 kDa), optionally inter-connected by disulfide bonds. There are two types of light chain: 2 and K. In humans, 2 and k light chains are similar, but only one type is present in each antibody. Heavy chains are classified as mu, delta, gamma, alpha, or epsilon, and define the antibody's isotype as IgM, IgD, IgG, IgA, and IgE, respectively. See generally, Fundamental Immunology Ch. 7 (Paul, W., ed., 2nd ed. Raven Press, N.Y. (1989)) (incorporated by reference in its entirety). The methods, uses, and compositions-for-use disclosed herein utilize IgG antibodies.

As used herein, the term “antigen-binding fragment” refers to a portion (or fragment) of an antibody that retains the antibodies binding specificity. Accordingly, as used herein, an antigen-binding fragment retains the six CDRs of the reference antibody.

As used herein, the term “effector function” refers to a biochemical event that results from the interaction of an antibody Fc region with an Fc receptor or another effector molecule (e.g., Fc receptor-Like (FcRL) molecules, complement component C1q, and Tripartite motif-containing protein 21 (TRIM21)). Effector functions include, but are not limited to, antibody dependent cell-mediated cytotoxicity (ADCC), antibody dependent cell-mediated phagocytosis (ADCP) and complement-dependent cellular cytotoxicity (CDC). The term “ADCC” or “antibody dependent cell-mediated cytotoxicity,” as used herein, refers to the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity. As is discussed herein, many embodiments of the present disclosure ablate ADCC activity entirely. The term “ADCP” or “antibody dependent cell-mediated phagocytosis,” as used herein, refers to the cell-mediated reaction wherein nonspecific cytotoxic cells that express FcγRs recognize bound antibody on a target cell and subsequently cause phagocytosis of the target cell. The term “CDC” or “complement-dependent cellular cytotoxicity,” as used herein, refers to an effector function which leads to the activation of the classical complement pathway, which is triggered by the binding of an antibody to an antigen on the target cell, which activates a series of cascades containing complement-related protein groups in blood.

As used herein, the terms “Fc,” “Fc region” or “Fc domain” are used interchangeably herein and refer to the polypeptide comprising the constant region of an antibody excluding, in some instances, the first constant region immunoglobulin domain (e.g., CHI) or a portion thereof, and in some cases, part of the hinge. For IgG, the Fc domain comprises immunoglobulin domains Cγ2 and Cγ3 (Cγ2 and Cγ3) and the lower hinge region between Cγ1 (Cγ1) and Cγ2 (Cγ2). In some embodiments, an Fc refers to a truncated CH1 domain, and CH2 and CH3 of an immunoglobulin. Although the boundaries of the Fc region may vary, the human IgG heavy chain Fc region is usually defined to include residues E216 or C226 or P230 to its carboxyl-terminus, wherein the numbering is according to the EU numbering. In some embodiments, as is more fully described herein, amino acid modifications are made to the Fc region, for example to alter binding to one or more FcγR receptors or to the FcRn receptor. In some embodiments, the Fc domain is derived from a human IgG1 heavy chain Fc domain. In some embodiments, the Fc domain is derived from a human IgG2 heavy chain Fc domain. The “EU format as set forth in Edelman” or “EU numbering” or “EU index” refers to the residue numbering of the human Fc domain as described in Edelman G M et al. (Proc. Natl. Acad. USA (1969), 63, 78-85, hereby entirely incorporated by reference).

As used herein, the terms “Fc fusion protein” and “immunoadhesin” are used interchangeably and refer to a protein comprising an Fc region, generally linked (optionally through a linker moiety, as described herein) to a different protein, such as to IL-15 and/or IL-15R, as described herein. In some instances, two Fc fusion proteins can form a homodimeric Fc fusion protein or a heterodimeric Fc fusion protein with the latter being preferred.

As used herein, the term “Fc variant” or “variant Fc” refers to a protein comprising an amino acid modification in an Fc domain. The Fc variants of the present invention are defined according to the amino acid modifications that compose them. Thus, for example, N434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. For all positions discussed in the present invention that relate to antibodies, unless otherwise noted, amino acid position numbering is according to the EU index. The modification can be an addition, deletion, or substitution

The terms “Fc gamma receptor,” “FcγR” and “FcgammaR,” as used herein, are used interchangeably and refer to any member of the family of proteins that bind the IgG antibody Fc region and is encoded by an FcγR gene. An FcγR may be from any organism. In some embodiments, the FcγR is a human FcγR. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes.

The term “FcRn” or “neonatal Fc Receptor,” as used herein, refers to a protein that binds the IgG antibody Fc region and is encoded at least in part by an FcRn gene. The FcRn may be from any organism. In some embodiments, the FcRn is a human FcRn. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants can be used to increase binding to the FcRn receptor, and in some cases, to increase serum half-life. In general, unless otherwise noted, the Fc monomers disclosed herein retain binding to the FcRn receptor (and, as noted below, can include amino acid variants to increase binding to the FcRn receptor).

As used herein, “IL-15,” “IL15” or “Interleukin 15” may be used interchangeably and refer to a four-a-helix protein belonging to a family of cytokines. IL-15 signals through a receptor complex composed of the IL-2/IL-15 receptor β (IL-15RB) (CD122) subunit. In some embodiments, the IL-15 protein comprises the polypeptide sequence set forth in SEQ ID NO:2 (full-length human IL-15). In some embodiments, the IL-15 protein comprises the polypeptide sequence set forth in SEQ ID NO: 1 (truncated or mature human IL-15). In some embodiments, the IL-15 protein comprises a polypeptide sequence selected from the group consisting of SEQ ID NO:1 and SEQ ID NO:2.

As used herein, “XENP24306,” “XmAb306,” “XmAb24306” may be used interchangeably and refer to an IL15-IL15Rα heterodimeric Fc-fusion protein, wherein the first monomer comprises the amino acid sequence of SEQ ID NO: 9 and the second monomer comprises the amino acid sequence of SEQ ID NO: 10.

The term “modification,” as used herein, refers to an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always referring to an amino acid coded for by DNA, e.g., the 20 amino acids that have codons in DNA and RNA.

The terms “nucleic acid,” “polynucleotide” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer.

The term “non-naturally occurring modification,” as used herein, refers to an amino acid modification that is not isotypic. For example, because none of the IgGs comprise a serine at position 434, the substitution 434S in IgG1, IgG2, or IgG4 (or hybrids thereof) is considered a non-naturally occurring modification.

The terms “patient,” “subject” and “individual” are used interchangeably herein and refer to a human in need to treatment. In some embodiments, the subject is in need of treatment of a blood cancer, such as multiple myeloma. The terms “treating” and “treatment,” as used herein, refer to reduction in severity and/or frequency of symptoms, elimination of symptoms and/or underlying cause, prevention of the occurrence of symptoms and/or their underlying cause, and improvement or remediation of damage.

As used herein, the terms “polypeptide,” “peptide” and “protein” are used interchangeably to refer to a polymer of amino acid residues. Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are known in the art.

The term “position,” as used herein, refers to a location in the sequence of a protein. Positions may be numbered sequentially, or according to an established format, for example the EU index for antibody numbering. A position may be defined relative to a reference sequence. In such cases, the reference sequence is provided for comparison purposes, and the heterodimeric protein of the disclosure (or a portion thereof) may comprise additional amino acid alterations (e.g., substitutions, insertions, and deletions) relative to the reference sequence. In some embodiments, the heterodimeric protein of the disclosure (or a portion thereof) does not comprise any additional amino acid alterations relative to the reference sequence.

The term “residue,” as used herein, refers to a position in a protein and its associated amino acid identity. For example, Asparagine 297 (also referred to as Asn297 or N297) is a residue at position 297 in a specific protein.

The terms “therapeutically effective amount” and “effective amount” are used interchangeably herein and refer to that amount of the therapeutic agent being administered, as a single agent or in combination with one or more additional agents, which will relieve to some extent one or more of the symptoms of the condition being treated. In some embodiments, the therapeutically effective amount is an amount sufficient to effect the beneficial or desired clinical results. With respect to the treatment of cancer, a therapeutically effective amount refers to that amount which has at least one of the following effects: palliate, ameliorate, stabilize, reverse, prevent, slow or delay the progression of (and/or symptoms associated with) of a blood cancer, such as multiple myeloma. The effective amounts that may be used in the present disclosure varies depending upon the manner of administration, the age, body weight, and general health of the subject. The appropriate amount and dosage regimen can be determined using routine skill in the art. For example, efficacy can be determined using the International Myeloma Working Group (IMWG) Uniform Response Criteria.

The terms “wild type” or “WT” are used interchangeably herein and refer to an amino acid sequence or a nucleotide sequence that is found in nature, including allelic variations. A WT protein has an amino acid sequence or is encoded by a nucleotide sequence that has not been intentionally modified.

The sequences referenced herein are provided in Table 1, infra. It is known in the art that during the processing and expression of Fc-containing proteins that the C-terminal lysine may be cleaved (also known in the art as C-terminal lysine clipping). Accordingly, for each sequence disclosed herein that contains a C-terminal lysine, the corresponding sequence without the C-terminal lysine (i.e. the C-terminal lysine cleavage product) is also contemplated. In some embodiments, the first monomer comprises a C-terminal lysine. In some embodiments, the first monomer lacks a C-terminal lysine. In some embodiments, the second monomer comprises a C-terminal lysine. In some embodiments, the second monomer lacks a C-terminal lysine.

It is also known in the art that the C-terminal cleavage process is imprecise and that additional C-terminal residues are cleaved. Accordingly, for each sequence disclosed herein that contains a C-terminal lysine, the corresponding sequence without the two C-terminal residues is also contemplated. In some embodiments, for each sequence disclosed herein that contains a C-terminal lysine, the corresponding sequence without the three C-terminal residues is also contemplated. In some embodiments, for each sequence disclosed herein that contains a C-terminal lysine, the corresponding sequence without the four C-terminal residues is also contemplated. In some embodiments, for each sequence disclosed herein that contains a C-terminal lysine, the corresponding sequence without the five C-terminal residues is also contemplated. In some embodiments, for each sequence disclosed herein that contains a C-terminal lysine, the corresponding sequence without the six C-terminal residues is also contemplated. In some embodiments, for each sequence disclosed herein that contains a C-terminal lysine, the corresponding sequence without the seven C-terminal residues is also contemplated. In some embodiments, for each sequence disclosed herein that contains a C-terminal lysine, the corresponding sequence without the eight C-terminal residues is also contemplated. In some embodiments, for each sequence disclosed herein that contains a C-terminal lysine, the corresponding sequence without the nine C-terminal residues is also contemplated. In some embodiments, for each sequence disclosed herein that contains a C-terminal lysine, the corresponding sequence without the ten C-terminal residues is also contemplated. In some embodiments, for each sequence disclosed herein that contains a C-terminal lysine, the corresponding sequence without the eleven C-terminal residues is also contemplated. In some embodiments, for each sequence disclosed herein that contains a C-terminal lysine, the corresponding sequence without the twelve C-terminal residues is also contemplated. In some embodiments, for each sequence disclosed herein that contains a C-terminal lysine, the corresponding sequence without the thirteen C-terminal residues is also contemplated. In some embodiments, for each sequence disclosed herein that contains a C-terminal lysine, the corresponding sequence without the fourteen C-terminal residues is also contemplated. In some embodiments, for each sequence disclosed herein that contains a C-terminal lysine, the corresponding sequence without the fifteen C-terminal residues is also contemplated. In some embodiments, the missing C-terminal residues are the result of engineering (e.g., expressing a polynucleotide missing the nucleotide sequences encoding one or more of the C-terminal residues).

TABLE 1
Compilation of amino acid sequences described in the present disclosure.

| | | |
|---|---|---|
| SEQ ID | Wild-type | NWVNVISDLKKIEDLIQSMHIDATLYTESDVHPS |
| NO: 1 | mature or | CKVTAMKCFLLELQVISLESGDASIHDTVENLIIL |
| | truncated IL-15 | ANNSLSSNGNVTESGCKECEELEEKNIKEFLQSF |
| | protein | VHIVQMFINTS |
| SEQ ID | Wild-type full- | MRISKPHLRSISIQCYLCLLLNSHFLTEAGIHVFIL |
| NO: 2 | length IL-15 | GCFSAGLPKTEANWVNVISDLKKIEDLIQSMHID |
| | protein | ATLYTESDVHPSCKVTAMKCFLLELQVISLESGD |
| | | ASIHDTVENLIILANNSLSSNGNVTESGCKECEEL |
| | | EEKNIKEFLQSFVHIVQMFINTS |
| SEQ ID | Wild-type full- | MAPRRARGCRTLGLPALLLLLLLRPPATRGITCPP |
| NO: 3 | length IL-15Rα | PMSVEHADIWVKSYSLYSRERYICNSGFKRKAG |
| | protein | TSSLTECVLNKATNVAHWTTPSLKCIRDPALVHQ |
| | | RPAPPSTVTTAGVTPQPESLSPSGKEPAASSPSSN |
| | | NTAATTAAIVPGSQLMPSKSPSTGTTEISSHESSH |
| | | GTPSQTTAKNWELTASASHQPPGVYPQGHSDTT |
| | | VAISTSTVLLCGLSAVSLLACYLKSRQTPPLASVE |
| | | MEAMEALPVTWGTSSRDEDLENCSHHL |
| SEQ ID | Sushi domain of | ITCPPPMSVEHADIWVKSYSLYSRERYICNSGFKR |
| NO: 4 | IL-15Rα protein | KAGTSSLTECVLNKATNVAHWTTPSLKCIR |
| SEQ ID | XmAb24306 IL-15 | NWVNVISDLKKIEDLIQSMHIDATLYTESNVHPS |
| NO: 5 | protein variant | CKVTAMKCFLLELQVISLESGDASIHDTVQDLIIL |
| | | ANNSLSSNGNVTESGCKECEELEEKNIKEFLQSF |
| | | VHIVQMFINTS |
| SEQ ID | XmAb24306 | EPKSSDKTHTCPPCPAPPVAGPSVFLFPPKPKDTL |
| NO: 6 | First IgG1 Fc | MISRTPEVTCVVVDVKHEDPEVKFNWYVDGVE |
| | domain (IL-15 | VHNAKTKPREEEYNSTYRVVSVLTVLHQDWLN |
| | monomer) | GKEYKCKVSNKALPAPIEKTISKAKGQPREPQVY |
| | | TLPPSREEMTKNQVSLTCDVSGFYPSDIAVEWES |
| | | DGQPENNYKTTPPVLDSDGSFFLYSKLTVDKSR |
| | | WEQGDVFSCSVLHEALHSHYTQKSLSLSPGK |
| SEQ ID | XmAb24306 | EPKSSDKTHTCPPCPAPPVAGPSVFLFPPKPKDTL |
| NO: 7 | Second IgG1 Fc | MISRTPEVTCVVVDVKHEDPEVKFNWYVDGVE |
| | domain (IL- | VHNAKTKPREEQYNSTYRVVSVLTVLHQDWLN |
| | 15Rα monomer) | GKEYKCKVSNKALPAPIEKTISKAKGQPREPQVY |
| | | TLPPSREQMTKNQVKLTCLVKGFYPSDIAVEWES |
| | | NGQPENNYKTTPPVLDSDGSFFLYSKLTVDKSR |
| | | WQQGNVFSCSVLHEALHSHYTQKSLSLSPGK |
| SEQ ID | Linker Domain | GGGGS |
| NO: 8 | for First and | |
| | Second | |
| | monomers | |
| SEQ ID | XmAb24306 | NWVNVISDLKKIEDLIQSMHIDATLYTESNVHPS |
| NO: 9 | First monomer | CKVTAMKCFLLELQVISLESGDASIHDTVQDLIIL |
| | (IL-15-first Fc | ANNSLSSNGNVTESGCKECEELEEKNIKEFLQSF |
| | domain | VHIVQMFINTSGGGGSEPKSSDKTHTCPPCPAPPV |
| | monomer) | AGPSVFLFPPKPKDTLMISRTPEVTCVVVDVKHE |
| | | DPEVKFNWYVDGVEVHNAKTKPREEEYNSTYR |
| | | VVSVLTVLHQDWLNGKEYKCKVSNKALPAPIEK |
| | | TISKAKGQPREPQVYTLPPSREEMTKNQVSLTCD |
| | | VSGFYPSDIAVEWESDGQPENNYKTTPPVLDSDG |
| | | SFFLYSKLTVDKSRWEQGDVFSCSVLHEALHSH |
| | | YTQKSLSLSPGK |
| SEQ ID | XmAb24306 | ITCPPPMSVEHADIWVKSYSLYSRERYICNSGFKR |
| NO: 10 | Second monomer | KAGTSSLTECVLNKATNVAHWTTPSLKCIRGGG |
| | (IL-15Rα-second | GSEPKSSDKTHTCPPCPAPPVAGPSVFLFPPKPKD |
| | Fc domain | TLMISRTPEVTCVVVDVKHEDPEVKFNWYVDGV |
| | monomer) | EVHNAKTKPREEQYNSTYRVVSVLTVLHQDWL |
| | | NGKEYKCKVSNKALPAPIEKTISKAKGQPREPQV |
| | | YTLPPSREQMTKNQVKLTCLVKGFYPSDIAVEW |
| | | ESNGQPENNYKTTPPVLDSDGSFFLYSKLTVDKS |
| | | RWQQGNVFSCSVLHEALHSHYTQKSLSLSPGK |
| SEQ ID | Anti-FcRH5 | KASQDVRNLVV |
| NO: 11 | CDR-L1 | |
| SEQ ID | Anti-FcRH5 | SGSYRYS |
| NO: 12 | CDR-L2 | |
| SEQ ID | Anti-FcRH5 | QQHYSPPYT |
| NO: 13 | CDR-L3 | |
| SEQ ID | Anti-FcRH5 | RFGVH |
| NO: 14 | CDR-H1 | |
| SEQ ID | Anti-FcRH5 | VIWRGGSTDYNAAFVS |
| NO: 15 | CDR-H2 | |
| SEQ ID | Anti-FcRH5 | HYYGSSDYALDN |
| NO: 16 | CDR-H3 | |
| SEQ ID | Anti-FcRH5 VL | DIQMTQSPSSLSASVGDRVTITCKASQDVRNLVV |
| NO: 17 | | WFQQKPGKAPKLLIYSGSYRYSGVPSRFSGSGSG |
| | | TDFTLTISSLQPEDFATYYCQQHYSPPYTFGQGT |
| | | KVEIK |
| SEQ ID | Anti-FcRH5 VH | EVQLVESGPGLVKPSETLSLTCTVSGFSLTRFGV |
| NO: 18 | | HWVRQPPGKGLEWLGVIWRGGSTDYNAAFVSR |
| | | LTISKDNSKNQVSLKLSSVTAADTAVYYCSNHY |
| | | YGSSDYALDNWGQGTLVTVSS |
| SEQ ID | Anti-FcRH5 | DIQMTQSPSSLSASVGDRVTITCKASQDVRNLVV |
| NO: 19 | Light Chain | WFQQKPGKAPKLLIYSGSYRYSGVPSRFSGSGSG |
| | | TDFTLTISSLQPEDFATYYCQQHYSPPYTFGQGT |
| | | KVEIKRTVAAPSVFIFPPSDEQLKSGTASVVCLLN |
| | | NFYPREAKVQWKVDNALQSGNSQESVTEQDSK |
| | | DSTYSLSSTLTLSKADYEKHKVYACEVTHQGLSS |
| | | PVTKSFNRGEC |
| SEQ ID | Anti-FcRH5 | EVQLVESGPGLVKPSETLSLTCTVSGFSLTRFGV |
| NO: 20 | Heavy Chain | HWVRQPPGKGLEWLGVIWRGGSTDYNAAFVSR |
| | | LTISKDNSKNQVSLKLSSVTAADTAVYYCSNHY |
| | | YGSSDYALDNWGQGTLVTVSSASTKGPSVFPLA |
| | | PSSKSTSGGTAALGCLVKDYFPEPVTVSWNSGAL |
| | | TSGVHTFPAVLQSSGLYSLSSVVTVPSSSLGTQTY |
| | | ICNVNHKPSNTKVDKKVEPKSCDKTHTCPPCPAP |
| | | ELLGGPSVFLFPPKPKDTLMISRTPEVTCVVVDVS |
| | | HEDPEVKFNWYVDGVEVHNAKTKPREEQYGST |
| | | YRVVSVLTVLHQDWLNGKEYKCKVSNKALPAPI |
| | | EKTISKAKGQPREPQVYTLPPSREEMTKNQVSLW |
| | | CLVKGFYPSDIAVEWESNGQPENNYKTTPPVLDS |
| | | DGSFFLYSKLTVDKSRWQQGNVFSCSVMHEALH |
| | | NHYTQKSLSLSPGK |
| SEQ ID | Anti-CD3 CDR- | KSSQSLLNSRTRKNYLA |
| NO: 21 | L1 | |
| SEQ ID | Anti-CD3 CDR- | WTSTRKS |
| NO: 22 | L2 | |
| SEQ ID | Anti-CD3 CDR- | HYYGSSDYALDN |
| NO: 23 | L3 | |
| SEQ ID | Anti-CD3 CDR- | SYYIH |
| NO: 24 | H1 | |
| SEQ ID | Anti-CD3 CDR- | WIYPENDNTKYNEKFKD |
| NO: 25 | H2 | |
| SEQ ID | Anti-CD3 CDR- | DGYSRYYFDY |
| NO: 26 | H3 | |
| SEQ ID | Anti-CD3 VL | DIVMTQSPDSLAVSLGERATINCKSSQSLLNSRTR |
| NO: 27 | | KNYLAWYQQKPGQSPKLLIYWTSTRKSGVPDRF |
| | | SGSGSGTDFTLTISSLQAEDVAVYYCKQSFILRTF |
| | | GQGTKVEIK |
| SEQ ID | Anti-CD3 VH | EVQLVQSGAEVKKPGASVKVSCKASGFTFTSYYI |
| NO: 28 | | HWVRQAPGQGLEWIGWIYPENDNTKYNEKFKD |
| | | RVTITADTSTSTAYLELSSLRSEDTAVYYCARDG |
| | | YSRYYFDYWGQGTLVTVSS |
| SEQ ID | Anti-CD3 Light | DIVMTQSPDSLAVSLGERATINCKSSQSLLNSRTR |
| NO: 29 | Chain | KNYLAWYQQKPGQSPKLLIYWTSTRKSGVPDRF |
| | | SGSGSGTDFTLTISSLQAEDVAVYYCKQSFILRTF |
| | | GQGTKVEIKRTVAAPSVFIFPPSDEQLKSGTASVV |
| | | CLLNNFYPREAKVQWKVDNALQSGNSQESVTE |
| | | QDSKDSTYSLSSTLTLSKADYEKHKVYACEVTH |
| | | QGLSSPVTKSFNRGEC |
| SEQ ID | Anti-CD3 Heavy | EVQLVQSGAEVKKPGASVKVSCKASGFTFTSYYI |
| NO: 30 | Chain | HWVRQAPGQGLEWIGWIYPENDNTKYNEKFKD |
| | | RVTITADTSTSTAYLELSSLRSEDTAVYYCARDG |
| | | YSRYYFDYWGQGTLVTVSSASTKGPSVFPLAPSS |
| | | KSTSGGTAALGCLVKDYFPEPVTVSWNSGALTS |
| | | GVHTFPAVLQSSGLYSLSSVVTVPSSSLGTQTYIC |
| | | NVNHKPSNTKVDKKVEPKSCDKTHTCPPCPAPE |
| | | LLGGPSVFLFPPKPKDTLMISRTPEVTCVVVDVS |
| | | HEDPEVKFNWYVDGVEVHNAKTKPREEQYGST |
| | | YRVVSVLTVLHQDWLNGKEYKCKVSNKALPAPI |
| | | EKTISKAKGQPREPQVYTLPPSREEMTKNQVSLS |
| | | CAVKGFYPSDIAVEWESNGQPENNYKTTPPVLD |
| | | SDGSFFLVSKLTVDKSRWQQGNVFSCSVMHEAL |
| | | HNHYTQKSLSLSPGK |

Method of Treatment with IL15-IL15Rα Heterodimeric Fc-Fusion Proteins and an FcRH5xCD3 Bispecific Antibody as Combination Therapy

The present disclosure relates to methods of treating a blood cancer in a subject in need thereof. In some embodiments, the method comprising administering to the subject a therapeutically effective amount of a heterodimeric Fc fusion protein that includes IL-15 and IL-15 receptor alpha (IL-15Rα) protein domains and an FcRH5xCD3 bispecific antibody. The present disclosure relates to methods for inducing the proliferation of CD8⁺ effector memory T cells in a subject suffering from a blood cancer, the method comprising administering to the subject an effective amount of a heterodimeric Fc fusion protein that includes IL-15 and IL-15 receptor alpha (IL-15Rα) protein domains and an FcRH5xCD3 bispecific antibody.

A first aspect of the present disclosure provides a method of treating a blood cancer as disclosed herein in a subject in need thereof, the method comprising administering to the subject an effective amount of (a) an IL15-IL15Rα heterodimeric Fc-fusion protein and (b) an FcRH5xCD3 bispecific antibody or a fragment thereof that binds both antigens. Without being bound by theory, the scientific rationale for this combination is that the T cell-mediated antitumor activity of the FcRH5xCD3 bispecific antibody may be enhanced by an increase in the proliferation, survival, and/or effector function of T cells upon exposure to an IL-15 agonist, such as an IL15-IL15Rα heterodimeric Fc-fusion protein, disclosed herein, like XmAb24306.

In a second aspect, the present disclosure provides use of a therapeutically effective amount of (a) an IL15-IL15Rα heterodimeric Fc-fusion protein and (b) an FcRH5xCD3 bispecific antibody or a fragment thereof that binds both antigens in the manufacture of one or more medicaments for the treatment of a blood cancer in a subject in need thereof.

In a third aspect, the present disclosure provides a therapeutically effective amount of (a) an IL15-IL15Rα heterodimeric Fc-fusion protein and (b) an FcRH5xCD3 bispecific antibody or a fragment thereof that binds both antigen for use in treating a blood cancer in a subject in need thereof.

In some embodiments, the IL15-IL15Rα heterodimeric Fc-fusion protein comprises: (a) a first fusion protein comprising an interleukin-15 (IL-15) protein covalently attached to the N-terminus of a first Fc domain via a first domain linker, wherein the IL-15 protein comprises the amino acid sequence of SEQ ID NO: 5 and wherein the first Fc domain is a variant of a human IgG1 Fc domain; and (b) a second fusion protein comprising an IL-15 receptor alpha (IL-15Rα) protein fragment covalently attached to the N-terminus of a second Fc domain via a second domain linker, wherein the IL-15Rα protein comprises the amino acid sequence of SEQ ID NO: 4 and wherein the second Fc domain is a variant of a human IgG1 Fc domain.

A blood cancer refers to an abnormal or excessive production of blood cells (e.g., white blood cells). Examples of blood cancers to be treated by the methods and uses disclosed herein include, but are not limited, leukemias, lymphomas, and myelomas. More particular non-limiting examples of such blood cancers include acute myeloid leukemia, adult acute lymphoblastic leukemia, chronic lymphocytic leukemia, non-Hodgkin's lymphoma, B-cell non-Hodgkin's lymphoma, and multiple myeloma. In some embodiments, the blood cancer is a relapsed or refractory. In some embodiments, the blood cancer is selected from the group consisting of leukemia, acute myeloid leukemia, adult acute lymphoblastic leukemia, chronic lymphocytic leukemia, lymphoma, non-Hodgkin's lymphoma, B-cell non-Hodgkin's lymphoma, and multiple myeloma. In some embodiments, the blood cancer is selected from the group consisting of leukemia, acute myeloid leukemia, adult acute lymphoblastic leukemia, chronic lymphocytic leukemia. In some embodiments, the blood cancer is selected from the group consisting of lymphoma, non-Hodgkin's lymphoma, B-cell non-Hodgkin's lymphoma. In some embodiments, the blood cancer is leukemia. In some embodiments, the blood cancer is acute myeloid leukemia. In some embodiments, the blood cancer is adult acute lymphoblastic leukemia. In some embodiments, the blood cancer is chronic lymphocytic leukemia. In some embodiments, the blood cancer is lymphoma. In some embodiments, the blood cancer is non-Hodgkin's lymphoma. In some embodiments, the blood cancer is B-cell non-Hodgkin's lymphoma. In some embodiments, the blood cancer is multiple myeloma. In some embodiments, the blood cancer is relapsed or refractory multiple myeloma. In some embodiments, the blood cancer is a blood cancer for which standard therapy does not exist, has proven to be ineffective or intolerable, or is considered inappropriate, or for whom a clinical trial of an investigational agent is a recognized standard of care.

Identifying a subject in need of such treatment can be in the judgment of a subject or a health care professional and can be subjective (e.g. opinion) or objective (e.g. measurable by a test or diagnostic method). Such treatment will be suitably administered to subjects suffering from, having, susceptible to, or at risk for the blood cancer.

IL15-IL15Rα heterodimeric Fc-fusion proteins are known in the art. See, e.g., WO 2018/071919, incorporated herein by reference. The use of IL15-IL15Rα heterodimeric Fc-fusion proteins for treating cancer is also known in the art. See, e.g., WO 2021/155042, incorporated herein by reference. XmAb24306 is an interleukin-15 (IL15)/IL15-receptor alpha (IL15Rα) fusion protein engineered with a heterodimeric Fc domain and half-life extension mutations (IL15/IL15Rα-Fc). By complexing IL15 and IL15Rα (CD215) on the same Fc domain, XmAb24306 selectively engages IL2RB (CD122) and the common gamma-chain (7) receptor (CD132) without engaging IL2Rα (CD25). Compared to IL2-based therapeutics, XmAb24306 is expected to selectively expand natural killer (NK) cells and CD8 T cells and have minimal impact on proliferation of regulatory T cells that are known to constitutively express IL2Rα. XmAb24306 has also been engineered to reduce affinity to the CD122/CD132 receptor complex and extend in vivo half-life to improve pharmacokinetics and prolong pharmacodynamic response. In XmAb24306, the IL-15 variant has engineered to have reduced binding affinity (compared with wild-type IL-15) towards IL-2/IL-15BY receptor complex with the goal of improving tolerability and extending pharmacokinetics by reducing acute toxicity, and ultimately promote antitumor immunity through IL-15 mediated signaling on CD8⁺ T cells and NK cells.

IL-15Rα protein is a transmembrane protein with very high affinity for IL-15 that facilitates IL-15 trafficking from the endoplasmic reticulum (ER) through the cytoplasm and presentation of IL-15/IL-15Rα complexes on the cell surface. As used herein, the term “sushi domain of IL-15Rα” refers to the truncated extracellular region of IL-15Rα or recombinant human IL-15 receptor α. In some embodiments, the IL-15Rα protein comprises a polypeptide sequence of SEQ ID NO:3 (full-length human IL-15Rα). In some embodiments, the IL-15Rα protein comprises a polypeptide sequence of SEQ ID NO:4 (sushi domain of human IL-15Rα).

In some embodiments, the IL15-IL15Rα heterodimeric Fc-fusion protein comprises: a) a first fusion protein comprising an interleukin-15 (IL-15) protein covalently attached to the N-terminus of a first Fc domain via a first domain linker, wherein the IL-15 protein comprises the amino acid sequence of SEQ ID NO: 5 and wherein the first Fc domain comprises the amino acid sequence of SEQ ID NO: 6; and b) a second fusion protein comprising an IL-15 receptor alpha (IL-15Rα) protein fragment covalently attached to the N-terminus of a second Fc domain via a second domain linker, wherein the IL-15Rα protein comprises the amino acid sequence of SEQ ID NO: 4 and wherein the second Fc domain comprises the amino acid sequence of SEQ ID NO: 7. In some embodiments, the first domain linker comprises the amino acid sequence of SEQ ID NO: 8. In some embodiments, the second domain linker comprises the amino acid sequence of SEQ ID NO: 8. In some embodiments, the first domain linker comprises the amino acid sequence of SEQ ID NO: 8 and the second domain linker comprises the amino acid sequence of SEQ ID NO: 8. In some embodiments, the first fusion protein comprises the amino acid sequence of SEQ ID NO: 9. In some embodiments, the second fusion protein comprises the amino acid sequence of SEQ ID NO: 10. In some embodiments, the heterodimeric protein comprises a first monomer comprising the amino acid sequence set forth in SEQ ID NO: 9, and a second monomer comprising the amino acid sequence set forth in SEQ ID NO: 10. In some embodiments, the IL15-IL15Rα heterodimeric Fc-fusion protein is XmAb24306.

The Fc receptor-like 5 (FcRH5, also known as FcRL5 or IRTA2) gene belongs to a family of six recently identified genes of the immunoglobulin superfamily (IgSF). The FcRH cDNAs encode type I transmembrane glycoproteins with multiple Ig-like extracellular domains and cytoplasmic domains containing consensus immunoreceptor tyrosine-based activating and/or inhibitory signaling motifs. FcRH5 has been implicated in enhanced proliferation and downstream isotype expression during the development of antigen-primed B-cells (Dement-Brown et al. J. Leukoc. Biol. 91:59-67, 2012). FcRH5 is selectively expressed in the B-cell lineage starting from pre-B cells, but unlike most B-cell markers, its expression is retained in plasma cells (Polson et al. 2006; Li et al. 2017). Analysis of FcRH5 RNA expression in 53 different normal human tissues demonstrated that FcRH5 expression is expressed exclusively in the B-cell lineage. The selective expression for B-lineage cells and tissues predicts a favorable safety profile for this target. Analogous to its expression in normal plasma cells, FcRH5 is expressed by myeloma tumor cells. FcRH5 expression was detected in all patient-derived myeloma tumor cells, suggesting near 100% prevalence in MM. Overall, the high prevalence in MM, the predicted favorable safety profile, and overexpression in high-risk patients indicate FcRH5 as a promising target for MM (Li et al. 2017).

CD3 (cluster of differentiation 3) is a protein complex and T cell co-receptor that plays a role in activating both the cytotoxic T cell (CD8⁺ naive T cells) and T helper cells (CD4⁺ naive T cells). CD3 comprises four distinct chains: a CD3γ chain, a CD38 chain, and two CD38 chains. These chains associate with the T-cell receptor (TCR) and the CD3-zeta (ζ-chain) to generate an activation signal in T lymphocytes. Accordingly, an FcRH5xCD3 bispecific antibody (e.g., cevostamab) can be used to target T cells to MM cells.

FcRH5xCD3 bispecific antibodies are known in the art. See, e.g., WO2016/205520, incorporated herein by reference. The use of FcRH5xCD3 bispecific antibodies for treating cancer, including multiple myeloma, is also known in the art. See, e.g., WO 2022/076462, incorporated herein by reference. In some embodiments, the FcRH5xCD3 bispecific antibody or a fragment thereof that binds both antigens comprises an anti-FcRH5 light chain variable region, an anti-FcRH5 heavy chain variable region, an anti-CD3 light chain variable region, and an anti-CD3 heavy chain variable region.

In some embodiments, the anti-FcRH5 light chain variable region comprises a light chain complementarity determining region-1 (CDR-L1) comprising the amino acid sequence KASQDVRNLVV (SEQ ID NO: 11); a CDR-L2 comprising the amino acid sequence SGSYRYS (SEQ ID NO: 12); and a CDR-L3 comprising the amino acid sequence QQHYSPPYT (SEQ ID NO: 13); and the anti-FcRH5 heavy chain variable region comprises a CDR-H1 comprising the amino acid sequence RFGVH (SEQ ID NO: 14); a CDR-H2 comprising the amino acid sequence VIWRGGSTDYNAAFVS (SEQ ID NO: 15); and a CDR-H3 comprising the amino acid sequence HYYGSSDYALDN (SEQ ID NO: 16), according to Kabat numbering. In some embodiments, the anti-FcRH5 light chain variable region comprises the amino acid sequence DIQMTQSPSSLSASVGDRVTITCKASQDVRNLVVWFQQKPGKAPKLLIYSGSY RYSGVPSRFSGSGSGTDFTLTISSLQPEDFATYYCQQHYSPPYTFGQGTKVEIK (SEQ ID NO: 17). In some embodiments, the anti-FcRH5 heavy chain variable region comprises the amino acid sequence EVOLVESGPGLVKPSETLSLTCTVSGFSLTRFGVHWVRQPPGKGLEWLGVIW RGGSTDYNAAFVSRLTISKDNSKNQVSLKLSSVTAADTAVYYCSNHYYGSSD YALDNWGQGTLVTVSS (SEQ ID NO: 18). In some embodiments, the anti-FcRH5 light chain comprises the amino acid sequence DIQMTQSPSSLSASVGDRVTITCKASQDVRNLVVWFQQKPGKAPKLLIYSGSY RYSGVPSRFSGSGSGTDFTLTISSLQPEDFATYYCQQHYSPPYTFGQGTKVEIK RTVAAPSVFIFPPSDEQLKSGTASVVCLLNNFYPREAKVQWKVDNALQSGNS QESVTEQDSKDSTYSLSSTLTLSKADYEKHKVYACEVTHQGLSSPVTKSFNR GEC (SEQ ID NO: 19). In some embodiments, the anti-FcRH5 heavy chain comprises the amino acid sequence EVOLVESGPGLVKPSETLSLTCTVSGFSLTRFGVHWVRQPPGKGLEWLGVIW RGGSTDYNAAFVSRLTISKDNSKNQVSLKLSSVTAADTAVYYCSNHYYGSSD YALDNWGQGTLVTVSSASTKGPSVFPLAPSSKSTSGGTAALGCLVKDYFPEP VTVSWNSGALTSGVHTFPAVLQSSGLYSLSSVVTVPSSSLGTQTYICNVNHKP SNTKVDKKVEPKSCDKTHTCPPCPAPELLGGPSVFLFPPKPKDTLMISRTPEVT CVVVDVSHEDPEVKFNWYVDGVEVHNAKTKPREEQYGSTYRVVSVLTVLH QDWLNGKEYKCKVSNKALPAPIEKTISKAKGQPREPQVYTLPPSREEMTKNQ VSLWCLVKGFYPSDIAVEWESNGQPENNYKTTPPVLDSDGSFFLYSKLTVDK SRWQQGNVFSCSVMHEALHNHYTQKSLSLSPGK (SEQ ID NO: 20).

In some embodiments, the anti-CD3 light chain variable region comprises a light chain complementarity determining region-1 (CDR-L1) comprising the amino acid sequence KSSQSLLNSRTRKNYLA (SEQ ID NO: 21); a CDR-L2 comprising the amino acid sequence WTSTRKS (SEQ ID NO: 22); and a CDR-L3 comprising the amino acid sequence HYYGSSDYALDN (SEQ ID NO: 23); and the anti-CD3 heavy chain variable region comprises a CDR-H1 comprising the amino acid sequence SYYIH (SEQ ID NO: 24); a CDR-H2 comprising the amino acid sequence WIYPENDNTKYNEKFKD (SEQ ID NO: 25); and a CDR-H3 comprising the amino acid sequence DGYSRYYFDY (SEQ ID NO: 26), according to Kabat numbering. In some embodiments, the anti-CD3 light chain variable region comprises the amino acid sequence DIVMTQSPDSLAVSLGERATINCKSSQSLLNSRTRKNYLAWYQQKPGQSPKL LIYWTSTRKSGVPDRFSGSGSGTDFTLTISSLQAEDVAVYYCKQSFILRTFGQG TKVEIK (SEQ ID NO: 27). In some embodiments, the anti-CD3 heavy chain variable region comprises the amino acid sequence EVQLVQSGAEVKKPGASVKVSCKASGFTFTSYYIHWVRQAPGQGLEWIGWI YPENDNTKYNEKFKDRVTITADTSTSTAYLELSSLRSEDTAVYYCARDGYSR YYFDYWGQGTLVTVSS (SEQ ID NO: 28). In some embodiments, the anti-CD3 light chain comprises the amino acid sequence DIVMTQSPDSLAVSLGERATINCKSSQSLLNSRTRKNYLAWYQQKPGQSPKL LIYWTSTRKSGVPDRFSGSGSGTDFTLTISSLQAEDVAVYYCKQSFILRTFGQG TKVEIKRTVAAPSVFIFPPSDEQLKSGTASVVCLLNNFYPREAKVQWKVDNA LQSGNSQESVTEQDSKDSTYSLSSTLTLSKADYEKHKVYACEVTHQGLSSPVT KSFNRGEC (SEQ ID NO: 29). In some embodiments, the anti-CD3 heavy chain comprises the amino acid sequence EVQLVQSGAEVKKPGASVKVSCKASGFTFTSYYIHWVRQAPGQGLEWIGWI YPENDNTKYNEKFKDRVTITADTSTSTAYLELSSLRSEDTAVYYCARDGYSR YYFDYWGQGTLVTVSSASTKGPSVFPLAPSSKSTSGGTAALGCLVKDYFPEP VTVSWNSGALTSGVHTFPAVLQSSGLYSLSSVVTVPSSSLGTQTYICNVNHKP SNTKVDKKVEPKSCDKTHTCPPCPAPELLGGPSVFLFPPKPKDTLMISRTPEVT CVVVDVSHEDPEVKFNWYVDGVEVHNAKTKPREEQYGSTYRVVSVLTVLH QDWLNGKEYKCKVSNKALPAPIEKTISKAKGQPREPQVYTLPPSREEMTKNQ VSLSCAVKGFYPSDIA VEWESNGQPENNYKTTPPVLDSDGSFFLVSKLTVDKS RWQQGNVFSCSVMHEALHNHYTQKSLSLSPGK (SEQ ID NO: 30).

In some embodiments, the FcRH5xCD3 bispecific antibody is cevostamab. Cevostamab is described in WHO Drug Information (International Nonproprietary Names for Pharmaceutical Substances), Recommended INN: List 84, Vol. 34, No. 3, published Sep. 9, 2020 (see pages 701-703). Cevostamab, which is also known in the art as BFCR4350A or RO7187797, is an Fc-engineered, humanized, full-length non-glycosylated IgG1 kappa T-cell-dependent bispecific antibody (TDB) that binds FcRH5 and CD3 and comprises an anti-FcRH5 arm comprising the heavy chain polypeptide sequence of SEQ ID NO: 20 and the light chain polypeptide sequence of SEQ ID NO: 19 and an anti-CD3 arm comprising the heavy chain polypeptide sequence of SEQ ID NO: 30 and the light chain polypeptide sequence of SEQ ID NO: 29. Cevostamab comprises a threonine to tryptophan amino acid substitution at position 366 on the heavy chain of the anti-FcRH5 arm (T366W) using EU numbering of Fc region amino acid residues and three amino acid substitutions (tyrosine to valine at position 407, threonine to serine at position 366, and leucine to alanine at position 368) on the heavy chain of the anti-CD3 arm (Y407V, T366S, and L368A) using EU numbering of Fc region amino acid residues to drive heterodimerization of the two arms (half-antibodies). Cevostamab also comprises an amino acid substitution (asparagine to glycine) at position 297 on each heavy chain (N297G) using EU numbering of Fc region amino acid residues, which results in a non-glycosylated antibody that has minimal binding to Fc (Fcγ) receptors and, consequently, prevents Fc-effector function.

In some embodiments, the FcRH5xCD3 bispecific antibody or a fragment thereof that binds both antigens is administered in combination with XmAb24306. In some embodiments, cevostamab is administered in combination with the IL15-IL15Rα heterodimeric Fc-fusion protein. In some embodiments, cevostamab or a fragment thereof that binds both antigens is administered in combination with the IL15-IL15Rα heterodimeric Fc-fusion protein. In some embodiments, cevostamab is administered in combination with XmAb24306. In some embodiments, cevostamab or a fragment thereof that binds both antigens is administered in combination with XmAb24306.

The FcRH5xCD3 bispecific antibody (e.g., cevostamab) or a fragment thereof that binds both antigens may be administered by any suitable route. In some embodiments, the FxRH5xCD3 bispecific antibody (e.g., cevostamab) or a fragment thereof that binds both antigens is administered parenterally. In some embodiments, the FxRH5xCD3 bispecific antibody (e.g., cevostamab) or a fragment thereof that binds both antigens is administered intravenously. In some embodiments, the FxRH5xCD3 bispecific antibody (e.g., cevostamab) or a fragment thereof that binds both antigens is administered subcutaneously. In some embodiments, cevostamab is administered intravenously. In some embodiments, cevostamab is administered subcutaneously.

In some embodiments, the FcRH5xCD3 bispecific antibody (e.g., cevostamab) or a fragment thereof that binds both antigens is administered systemically. In some embodiments, the FcRH5xCD3 bispecific antibody (e.g., cevostamab) or a fragment thereof that binds both antigens is administered as a composition comprising a pharmaceutically acceptable buffer. Suitable carriers and their formulations are described, for example, in Remington's Pharmaceutical Sciences by E. W. Martin. In some embodiments, the FcRH5xCD3 bispecific antibody (e.g., cevostamab) or a fragment thereof that binds both antigens is provided in a dosage form that is suitable for parenteral (e.g., intravenous) administration.

The amount of the FcRH5xCD3 bispecific antibody (e.g., cevostamab) or a fragment thereof that binds both antigens to be administered in combination with the heterodimeric protein of the disclosure (or combinations thereof) varies depending upon the manner of administration, the age and body weight of the patient, and the clinical symptoms of the cancer to be treated. A physician will be able to determine the adequate dosage of the FcRH5xCD3 bispecific antibody (e.g., cevostamab) or a fragment thereof that binds both antigens to administer in combination with the heterodimeric protein of the disclosure.

In some embodiments, the dosage of the FcRH5xCD3 bispecific antibody (e.g., cevostamab) is about 0.15 mg to 198 mg. In some embodiments, the dosage of the FcRH5xCD3 bispecific antibody (e.g., cevostamab) is about 132 mg to about 198 mg. In some embodiments, the dosage of the FcRH5xCD3 bispecific antibody (e.g., cevostamab) is 132 mg to 198 mg. In some embodiments, the dosage of the FcRH5xCD3 bispecific antibody (e.g., cevostamab) is about 132 mg. In some embodiments, the dosage of the FcRH5xCD3 bispecific antibody (e.g., cevostamab) is about 132 mg every week. In some embodiments, the dosage of the FcRH5xCD3 bispecific antibody (e.g., cevostamab) is about 132 mg every two weeks. In some embodiments, the dosage of the FcRH5xCD3 bispecific antibody (e.g., cevostamab) is about 132 mg every three weeks. In some embodiments, the dosage of the FcRH5xCD3 bispecific antibody (e.g., cevostamab) is about 132 mg every four weeks. In some embodiments, the dosage of the FcRH5xCD3 bispecific antibody (e.g., cevostamab) is about 132 mg every five weeks.

In some embodiments, the dosage of the FcRH5xCD3 bispecific antibody (e.g., cevostamab) is 132 mg. In some embodiments, the dosage of the FcRH5xCD3 bispecific antibody (e.g., cevostamab) is 132 mg every week. In some embodiments, the dosage of the FcRH5xCD3 bispecific antibody (e.g., cevostamab) is 132 mg every two weeks. In some embodiments, the dosage of the FcRH5xCD3 bispecific antibody (e.g., cevostamab) is 132 mg every three weeks. In some embodiments, the dosage of the FcRH5xCD3 bispecific antibody (e.g., cevostamab) is 132 mg every four weeks. In some embodiments, the dosage of the FcRH5xCD3 bispecific antibody (e.g., cevostamab) is 132 mg every five weeks.

In some embodiments, the dosage of the FcRH5xCD3 bispecific antibody (e.g., cevostamab) is about 160 mg. In some embodiments, the dosage of the FcRH5xCD3 bispecific antibody (e.g., cevostamab) is about 160 mg every week. In some embodiments, the dosage of the FcRH5xCD3 bispecific antibody (e.g., cevostamab) is about 160 mg every two weeks. In some embodiments, the dosage of the FcRH5xCD3 bispecific antibody (e.g., cevostamab) is about 160 mg every three weeks. In some embodiments, the dosage of the FcRH5xCD3 bispecific antibody (e.g., cevostamab) is about 160 mg every four weeks. In some embodiments, the dosage of the FcRH5xCD3 bispecific antibody (e.g., cevostamab) is about 160 mg every five weeks.

In some embodiments, the dosage of the FcRH5xCD3 bispecific antibody (e.g., cevostamab) is 160 mg. In some embodiments, the dosage of the FcRH5xCD3 bispecific antibody (e.g., cevostamab) is 160 mg every week. In some embodiments, the dosage of the FcRH5xCD3 bispecific antibody (e.g., cevostamab) is 160 mg every two weeks. In some embodiments, the dosage of the FcRH5xCD3 bispecific antibody (e.g., cevostamab) is 160 mg every three weeks. In some embodiments, the dosage of the FcRH5xCD3 bispecific antibody (e.g., cevostamab) is 160 mg every four weeks. In some embodiments, the dosage of the FcRH5xCD3 bispecific antibody (e.g., cevostamab) is 160 mg every five weeks.

In some embodiments, the dosage of the FcRH5xCD3 bispecific antibody (e.g., cevostamab) is about 198 mg. In some embodiments, the dosage of the FcRH5xCD3 bispecific antibody (e.g., cevostamab) is about 198 mg every week. In some embodiments, the dosage of the FcRH5xCD3 bispecific antibody (e.g., cevostamab) is about 198 mg every two weeks. In some embodiments, the dosage of the FcRH5xCD3 bispecific antibody (e.g., cevostamab) is about 198 mg every three weeks. In some embodiments, the dosage of the FcRH5xCD3 bispecific antibody (e.g., cevostamab) is about 198 mg every four weeks. In some embodiments, the dosage of the FcRH5xCD3 bispecific antibody (e.g., cevostamab) is about 198 mg every five weeks.

In some embodiments, the dosage of the FcRH5xCD3 bispecific antibody (e.g., cevostamab) is 198 mg. In some embodiments, the dosage of the FcRH5xCD3 bispecific antibody (e.g., cevostamab) is 198 mg every week. In some embodiments, the dosage of the FcRH5xCD3 bispecific antibody (e.g., cevostamab) is 198 mg every two weeks. In some embodiments, the dosage of the FcRH5xCD3 bispecific antibody (e.g., cevostamab) is 198 mg every three weeks. In some embodiments, the dosage of the FcRH5xCD3 bispecific antibody (e.g., cevostamab) is 198 mg every four weeks. In some embodiments, the dosage of the FcRH5xCD3 bispecific antibody (e.g., cevostamab) is 198 mg every five weeks.

In some embodiments, the dosage of cevostamab is about 0.15 mg to 198 mg. In some embodiments, the dosage of cevostamab is about 132 mg to about 198 mg. In some embodiments, the dosage of cevostamab is 132 mg to 198 mg. In some embodiments, the dosage of cevostamab is about 132 mg. In some embodiments, the dosage of cevostamab is about 132 mg every week. In some embodiments, the dosage of cevostamab is about 132 mg every two weeks. In some embodiments, the dosage of cevostamab is about 132 mg every three weeks. In some embodiments, the dosage of cevostamab is about 132 mg every four weeks. In some embodiments, the dosage of cevostamab is about 132 mg every five weeks.

In some embodiments, the dosage of cevostamab is 132 mg. In some embodiments, the dosage of cevostamab is 132 mg every week. In some embodiments, the dosage of cevostamab is 132 mg every two weeks. In some embodiments, the dosage of cevostamab is 132 mg every three weeks. In some embodiments, the dosage of cevostamab is 132 mg every four weeks. In some embodiments, the dosage of cevostamab is 132 mg every five weeks.

In some embodiments, the dosage of cevostamab is about 160 mg. In some embodiments, the dosage of cevostamab is about 160 mg every week. In some embodiments, the dosage of cevostamab is about 160 mg every two weeks. In some embodiments, the dosage of cevostamab is about 160 mg every three weeks. In some embodiments, the dosage of cevostamab is about 160 mg every four weeks. In some embodiments, the dosage of cevostamab is about 160 mg every five weeks.

In some embodiments, the dosage of cevostamab is 160 mg. In some embodiments, the dosage of cevostamab is 160 mg every week. In some embodiments, the dosage of cevostamab is 160 mg every two weeks. In some embodiments, the dosage of cevostamab is 160 mg every three weeks. In some embodiments, the dosage of cevostamab is 160 mg every four weeks. In some embodiments, the dosage of cevostamab is 160 mg every five weeks.

In some embodiments, the dosage of cevostamab is about 198 mg. In some embodiments, the dosage of cevostamab is about 198 mg every week. In some embodiments, the dosage of cevostamab is about 198 mg every two weeks. In some embodiments, the dosage of cevostamab is about 198 mg every three weeks. In some embodiments, the dosage of cevostamab is about 198 mg every four weeks. In some embodiments, the dosage of cevostamab is about 198 mg every five weeks.

In some embodiments, the dosage of cevostamab is 198 mg. In some embodiments, the dosage of cevostamab is 198 mg every week. In some embodiments, the dosage of cevostamab is 198 mg every two weeks. In some embodiments, the dosage of cevostamab is 198 mg every three weeks. In some embodiments, the dosage of cevostamab is 198 mg every four weeks. In some embodiments, the dosage of cevostamab is 198 mg every five weeks.

In some embodiments, the FcRH5xCD3 bispecific antibody (e.g., cevostamab) or a fragment thereof that binds both antigens is administered daily, i.e., every 24 hours. In some embodiments, the FcRH5xCD3 bispecific antibody (e.g., cevostamab) or a fragment thereof that binds both antigens is administered weekly, i.e., once per week (Q1W). In some embodiments, the FcRH5xCD3 bispecific antibody (e.g., cevostamab) or a fragment thereof that binds both antigens is administered once every two weeks, i.e., once every 14 days (Q2W). In some embodiments, the FcRH5xCD3 bispecific antibody (e.g., cevostamab) or a fragment thereof that binds both antigens is administered once every three weeks, i.e., once every 21 days (Q3W). In some embodiments, the FcRH5xCD3 bispecific antibody (e.g., cevostamab) or a fragment thereof that binds both antigens is administered once every four weeks, i.e., once every 28 days (Q4W). In some embodiments, the FcRH5xCD3 bispecific antibody (e.g., cevostamab) or a fragment thereof that binds both antigens is administered once every five weeks (Q5W). In some embodiments, the FcRH5xCD3 bispecific antibody (e.g., cevostamab) or a fragment thereof that binds both antigens is administered once every six weeks (Q6W). In some embodiments, the FcRH5xCD3 bispecific antibody (e.g., cevostamab) or a fragment thereof that binds both antigens is administered once every month. In some embodiments, the FcRH5xCD3 bispecific antibody (e.g., cevostamab) or a fragment thereof that binds both antigens is administered intravenously according to a frequency disclosed herein.

In some embodiments, the FcRH5xCD3 bispecific antibody (e.g., cevostamab) or a fragment thereof that binds both antigens is administered at any of the above frequencies in one or more cycles. In some embodiments, the FcRH5xCD3 bispecific antibody (e.g., cevostamab) or a fragment thereof that binds both antigens is administered at any of the above frequencies in 1, 2, 3, 4, 5, 6, 7, 8, 9, 10, 11, 12, 13, 14, 15, 16, 17, 18, 19 or 20 cycles. In some embodiments, the FcRH5xCD3 bispecific antibody (e.g., cevostamab) or a fragment thereof that binds both antigens is administered at a frequency of Q1W in one or more cycles. In some embodiments, the FcRH5xCD3 bispecific antibody (e.g., cevostamab) or a fragment thereof that binds both antigens is administered at a frequency of Q1W in 1, 2, 3, 4, 5, 6, 7, 8, 9, 10, 11, 12, 13, 14, 15, 16, 17, 18, 19 or 20 cycles. In some embodiments, the FcRH5xCD3 bispecific antibody (e.g., cevostamab) or a fragment thereof that binds both antigens is administered at a frequency of Q2W in one or more cycles. In some embodiments, the FcRH5xCD3 bispecific antibody (e.g., cevostamab) or a fragment thereof that binds both antigens is administered at a frequency of Q1W for four cycles. In some embodiments, the FcRH5xCD3 bispecific antibody (e.g., cevostamab) or a fragment thereof that binds both antigens is administered at a frequency of Q2W in 1, 2, 3, 4, 5, 6, 7, 8, 9, 10, 11, 12, 13, 14, 15, 16, 17, 18, 19 or 20 cycles. In some embodiments, the FcRH5xCD3 bispecific antibody is administered at a frequency of Q2W for six cycles. In some embodiments, the FcRH5xCD3 bispecific antibody (e.g., cevostamab) or a fragment thereof that binds both antigens is administered at a frequency of Q3W in one or more cycles. In some embodiments, the FcRH5xCD3 bispecific antibody (e.g., cevostamab) or a fragment thereof that binds both antigens is administered at a frequency of Q3W in 1, 2, 3, 4, 5, 6, 7, 8, 9, 10, 11, 12, 13, 14, 15, 16, 17, 18, 19 or 20 cycles. In some embodiments, the FcRH5xCD3 bispecific antibody (e.g., cevostamab) or a fragment thereof that binds both antigens is administered at a frequency of Q4W in one or more cycles. In some embodiments, the FcRH5xCD3 bispecific antibody (e.g., cevostamab) or a fragment thereof that binds both antigens is administered at a frequency of Q4W in 1, 2, 3, 4, 5, 6, 7, 8, 9, 10, 11, 12, 13, 14, 15, 16, 17, 18, 19 or 20 cycles. In some embodiments, each of the cycles is a 28-day cycle.

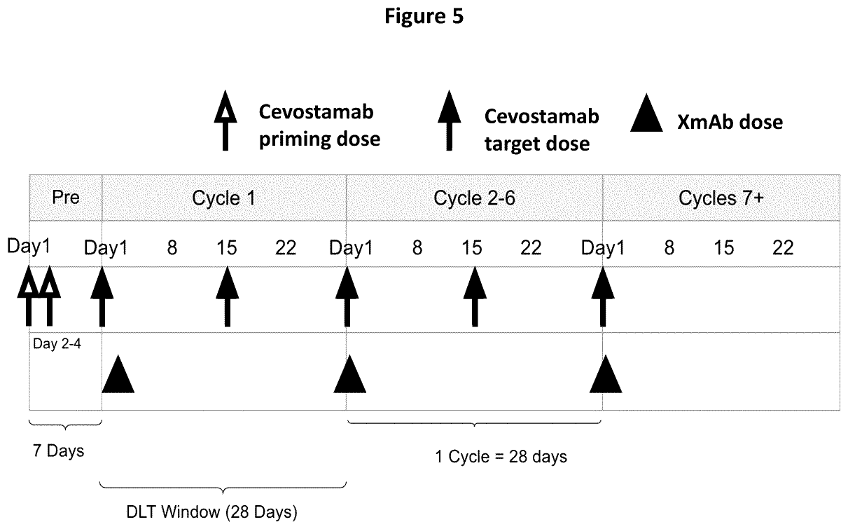

In some embodiments, the subject will be administered one or more priming dose of the FcRH5xCD3 bispecific antibody (e.g., cevostamab) or a fragment thereof that binds both antigens during a pre-phase before the first treatment cycle. In some embodiments, the pre-phase is seven days. See, e.g., FIG. 5. In some embodiments, the priming dose is 3.6 mg. In some embodiments, the priming dose is about 3.6 mg. In some embodiments, the subject will be administered two priming doses on the FcRH5xCD3 bispecific antibody (e.g., cevostamab) or a fragment thereof that binds both antigens. In some embodiments, 3.6 mg of the FcRH5xCD3 bispecific antibody (e.g., cevostamab) or a fragment thereof that binds both antigens is administered between the two priming doses (i.e., the amount administered in the first priming dose and the amount administered in the second priming dose add up to 3.6 mg). In some embodiments, about 3.6 mg of the FcRH5xCD3 bispecific antibody (e.g., cevostamab) is administered between the two priming doses (i.e., the amount administered in the first priming dose and the amount administered in the second priming dose add up to about 3.6 mg). In some embodiments, the first priming dose is administered on pre-phase Day 1. In some embodiments, the second priming dose is administered between pre-phase Days 2-4. In some embodiments, the minimum interval between the end of the first priming dose and the initiation of the second priming dose is 20 hours. In some embodiments, the first priming dose is 0.3 mg of the FcRH5xCD3 bispecific antibody (e.g., cevostamab). In some embodiments, the first priming dose is about 0.3 mg of the FcRH5xCD3 bispecific antibody (e.g., cevostamab). In some embodiments, the second priming dose is 3.3 mg of the FcRH5xCD3 bispecific antibody (e.g., cevostamab). In some embodiments, the second priming dose is about 3.3 mg of the FcRH5xCD3 bispecific antibody (e.g., cevostamab). Without being bound by theory, splitting the 3.6 mg dose over consecutive days (0.3 mg and 3.3 mg) is thought to reduce of the overall Cycle 1 risk of cytokine-release syndrome (CRS) compared with the 3.6 mg dose in the single dose priming regimen.

In some embodiments, the FcRH5xCD3 bispecific antibody (e.g., cevostamab) or a fragment thereof that binds both antigens is administered Q2W for the first 24 weeks and then Q4W after the first 24 weeks. In some embodiments, each cycle is a 4-week cycle. In some embodiments, the FcRH5xCD3 bispecific antibody (e.g., cevostamab) or a fragment thereof that binds both antigens is administered Q2W for six four-week cycles and administered Q4W in the seventh and any subsequent four-week cycle. In some embodiments, the FcRH5xCD3 bispecific antibody (e.g., cevostamab) or a fragment thereof that binds both antigens is administered on Days 1 and 15 for six four-week cycles and administered on Day 1 in the seventh and any subsequent four-week cycle. In some embodiments, the FcRH5xCD3 bispecific antibody (e.g., cevostamab) or a fragment thereof that binds both antigens is administered at a dose of 132 mg. In some embodiments, the FcRH5xCD3 bispecific antibody (e.g., cevostamab) or a fragment thereof that binds both antigens is administered at a dose of about 132 mg.

In some embodiments, the patient is administered a first priming dose of the FcRH5xCD3 bispecific antibody (e.g., cevostamab) or a fragment thereof that binds both antigens on pre-phase Day 1, a second priming dose between pre-phase Days 2-4 and, thereafter, Q2W for six four-week cycles and Q4W for the seventh and any subsequent four-week cycles. In some embodiments, the patient is administered a first priming dose of the FcRH5xCD3 bispecific antibody (e.g., cevostamab) or a fragment thereof that binds both antigens on pre-phase Day 1, a second priming dose between pre-phase Days 2-4 and, thereafter, Days 1 and 15 for six four-week cycles and on Day 1 for the seventh and any subsequent four-week cycles. In some embodiments, the patient is administered a first priming dose of 0.3 mg of the FcRH5xCD3 bispecific antibody (e.g., cevostamab) on pre-phase Day 1, a second priming dose of 3.3 mg of the FcRH5xCD3 bispecific antibody (e.g., cevostamab) between pre-phase Days 2-4 and, thereafter, at a dose of 132 mg of the FcRH5xCD3 bispecific antibody (e.g., cevostamab) on Days 1 and 15 for six four-week cycles and on Day 1 for the seventh and any subsequent four-week cycles.

The IL15-IL15Rα heterodimeric Fc-fusion protein (e.g., XmAb24306) may be administered by any suitable route. In some embodiments, the IL15-IL15Rα heterodimeric Fc-fusion protein (e.g., XmAb24306) is administered parenterally. In some embodiments, the parenteral administration is intravenous. In some embodiments, XmAb24306 is administered intravenously.

In some embodiments, the IL15-IL15Rα heterodimeric Fc-fusion protein (e.g., XmAb24306) is administered systemically. In some embodiments, the IL15-IL15Rα heterodimeric Fc-fusion protein (e.g., XmAb24306) is administered as a composition comprising a pharmaceutically acceptable buffer. Suitable carriers and their formulations are described, for example, in Remington's Pharmaceutical Sciences by E. W. Martin. In some embodiments, the heterodimeric protein is provided in a dosage form that is suitable for parenteral (e.g., intravenous) administration.

The amount of the IL15-IL15Rα heterodimeric Fc-fusion protein to be administered in combination with FcRH5xCD3 bispecific antibody (e.g., cevostamab) or a fragment thereof that binds both antigens varies depending upon the manner of administration, the age and body weight of the patient, and the clinical symptoms of the cancer to be treated. A physician will be able to determine the adequate dosage of the IL15-IL15Rα heterodimeric Fc-fusion protein to administer in combination with the FcRH5xCD3 bispecific antibody (e.g., cevostamab) or a fragment thereof that binds both antigens.

In certain embodiments, the dosage of the IL15-IL15Rα heterodimeric Fc-fusion protein (e.g., XmAb24306) may vary from between about 0.0001 mg protein/kg body weight to about 5 mg protein/kg body weight; or from about 0.001 mg/kg body weight to about 4 mg/kg body weight or from about 0.005 mg/kg body weight to about 1 mg/kg body weight or from about 0.005 mg/kg body weight to about 0.3 mg/kg body weight or from about 0.005 mg/kg body weight to about 0.2 mg/kg body weight or from about 0.005 mg/kg body weight to about 0.02 mg/kg body weight. In some embodiments, this dose of the IL15-IL15Rα heterodimeric Fc-fusion protein (e.g., XmAb24306) may be about 0.0001, about 0.00025, about 0.0003, about 0.0005, about 0.001, about 0.003, about 0.005, about 0.008, about 0.01, about 0.015, about 0.02, about 0.03, about 0.04, about 0.05, about 0.06, about 0.07, about 0.08, about 0.09, about 0.1, about 0.12, about 0.135, about 0.15, about 0.16, about 0.2, about 0.2025, about 0.24, about 0.25, about 0.3, about 0.32, about 0.35, about 0.4, about 0.45, about 0.5, about 0.55, or about 0.6 mg/kg body weight. In some embodiments, the dose of the IL15-IL15Rα heterodimeric Fc-fusion protein (e.g., XmAb24306) is about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.1 mg/kg, about 0.12 mg/kg, about 0.16 mg/kg, about 0.2 mg/kg, about 0.24 mg/kg and about 0.32 mg/kg body weight. In some embodiments, the dosage of the IL15-IL15Rα heterodimeric Fc-fusion protein (e.g., XmAb24306) is about 0.0025 mg/kg body weight. In some embodiments, the dosage of the IL15-IL15Rα heterodimeric Fc-fusion protein (e.g., XmAb24306) is about 0.01 mg/kg body weight. In some embodiments, the dosage of the IL15-IL15Rα heterodimeric Fc-fusion protein (e.g., XmAb24306) is about 0.015 mg/kg body weight. In some embodiments, the dosage of the IL15-IL15Rα heterodimeric Fc-fusion protein (e.g., XmAb24306) is about 0.02 mg/kg body weight. In some embodiments, the dosage of the IL15-IL15Rα heterodimeric Fc-fusion protein (e.g., XmAb24306) is about 0.03 mg/kg body weight. In some embodiments, the dosage of the IL15-IL15Rα heterodimeric Fc-fusion protein (e.g., XmAb24306) is about 0.04 mg/kg body weight. In some embodiments, the dosage of the IL15-IL15Rα heterodimeric Fc-fusion protein (e.g., XmAb24306) is about 0.06 mg/kg body weight. In some embodiments, the dosage of the IL15-IL15Rα heterodimeric Fc-fusion protein (e.g., XmAb24306) is about 0.08 mg/kg body weight. In some embodiments, the dosage of the IL15-IL15Rα heterodimeric Fc-fusion protein (e.g., XmAb24306) is about 0.09 mg/kg body weight. In some embodiments, the dosage of the IL15-IL15Rα heterodimeric Fc-fusion protein (e.g., XmAb24306) is about 0.12 mg/kg body weight. In some embodiments, the dosage of the IL15-IL15Rα heterodimeric Fc-fusion protein (e.g., XmAb24306) is about 0.135 mg/kg body weight. In some embodiments, the dosage of the IL15-IL15Rα heterodimeric Fc-fusion protein (e.g., XmAb24306) is about 0.16 mg/kg body weight. In some embodiments, the dosage of the IL15-IL15Rα heterodimeric Fc-fusion protein (e.g., XmAb24306) is about 0.2025 mg/kg body weight. In some embodiments, the dosage of the IL15-IL15Rα heterodimeric Fc-fusion protein (e.g., XmAb24306) is about 0.24 mg/kg body weight. In some embodiments, the dosage of the IL15-IL15Rα heterodimeric Fc-fusion protein (e.g., XmAb24306) is about 0.32 mg/kg body weight. In some embodiments, the IL15-IL15Rα heterodimeric Fc-fusion protein (e.g. XmAb24306) is administered at a dose selected from the group consisting of about 0.01 mg/kg, about 0.02 mg/kg, about 0.04 mg/kg, about 0.06 mg/kg, about 0.09 mg/kg, about 0.135 mg/kg, and about 0.2025 mg/kg body weight. In some embodiments, the IL15-IL15Rα heterodimeric Fc-fusion protein (e.g. XmAb24306) is administered at a dose selected from the group consisting of about 0.01 mg/kg, about 0.02 mg/kg, about 0.04 mg/kg, about 0.06 mg/kg, about 0.09 mg/kg, and about 0.12 mg/kg body weight. In some embodiments, the IL15-IL15Rα heterodimeric Fc-fusion protein (e.g., XmAb24306) is administered by IV infusion according to these dosages.

In certain embodiments, the dosage of the IL15-IL15Rα heterodimeric Fc-fusion protein (e.g., XmAb24306) may vary from between 0.0001 mg protein/kg body weight to 5 mg protein/kg body weight; or from 0.001 mg/kg body weight to 4 mg/kg body weight or from 0.005 mg/kg body weight to 1 mg/kg body weight or from 0.005 mg/kg body weight to 0.3 mg/kg body weight or from 0.005 mg/kg body weight to 0.2 mg/kg body weight or from 0.005 mg/kg body weight to 0.02 mg/kg body weight. In some embodiments, this dose of the IL15-IL15Rα heterodimeric Fc-fusion protein (e.g., XmAb24306) may be 0.0001, 0.0003, 0.0005, 0.001, 0.003, 0.005, 0.008, 0.01, 0.015, 0.02, 0.03, 0.05, 0.08, 0.1, 0.15, 0.2, 0.25, 0.3, 0.35, 0.4, 0.45, 0.5, 0.55, or 0.6 mg/kg body weight. In some embodiments, the dose of the IL15-IL15Rα heterodimeric Fc-fusion protein (e.g., XmAb24306) is selected from the group consisting of 0.0025 mg/kg, 0.005 mg/kg, 0.01 mg/kg, 0.015 mg/kg, 0.02 mg/kg, 0.025 mg/kg, 0.03 mg/kg, 0.04 mg/kg, 0.05 mg/kg, 0.06 mg/kg, 0.08 mg/kg, 0.09 mg/kg, 0.10 mg/kg, 0.12 mg/kg, 0.135 mg/kg, 0.16 mg/kg, 0.20 mg/kg, 0.2025 mg/kg, 0.24 mg/kg and 0.32 mg/kg body weight. In some embodiments, the dosage of the IL15-IL15Rα heterodimeric Fc-fusion protein (e.g., XmAb24306) is 0.0025 mg/kg body weight. In some embodiments, the dosage of the IL15-IL15Rα heterodimeric Fc-fusion protein (e.g., XmAb24306) is 0.01 mg/kg body weight. In some embodiments, the dosage of the IL15-IL15Rα heterodimeric Fc-fusion protein (e.g., XmAb24306) is 0.015 mg/kg body weight. In some embodiments, the dosage of the IL15-IL15Rα heterodimeric Fc-fusion protein (e.g., XmAb24306) is 0.02 mg/kg body weight. In some embodiments, the dosage of the IL15-IL15Rα heterodimeric Fc-fusion protein (e.g., XmAb24306) is 0.03 mg/kg body weight. In some embodiments, the dosage of the IL15-IL15Rα heterodimeric Fc-fusion protein (e.g., XmAb24306) is 0.04 mg/kg body weight. In some embodiments, the dosage of the IL15-IL15Rα heterodimeric Fc-fusion protein is 0.06 mg/kg body weight. In some embodiments, the dosage of the IL15-IL15Rα heterodimeric Fc-fusion protein (e.g., XmAb24306) is 0.08 mg/kg body weight. In some embodiments, the dosage of the IL15-IL15Rα heterodimeric Fc-fusion protein (e.g., XmAb24306) is 0.09 mg/kg body weight. In some embodiments, the dosage of the IL15-IL15Rα heterodimeric Fc-fusion protein (e.g., XmAb24306) is 0.12 mg/kg body weight. In some embodiments, the dosage of the IL15-IL15Rα heterodimeric Fc-fusion protein is 0.135 mg/kg body weight. In some embodiments, the dosage of the IL15-IL15Rα heterodimeric Fc-fusion protein (e.g., XmAb24306) is 0.16 mg/kg body weight. In some embodiments, the dosage of the IL15-IL15Rα heterodimeric Fc-fusion protein (e.g., XmAb24306) is 0.2025 mg/kg body weight. In some embodiments, the dosage of the IL15-IL15Rα heterodimeric Fc-fusion protein (e.g., XmAb24306) is 0.24 mg/kg body weight. In some embodiments, the dosage of the IL15-IL15Rα heterodimeric Fc-fusion protein (e.g., XmAb24306) is 0.32 mg/kg body weight. In some embodiments, the IL15-IL15Rα heterodimeric Fc-fusion protein (e.g. XmAb24306) is administered at a dose selected from the group consisting of 0.01 mg/kg, 0.02 mg/kg, 0.04 mg/kg, 0.06 mg/kg, 0.09 mg/kg, 0.135 mg/kg, and 0.2025 mg/kg body weight. In some embodiments, the IL15-IL15Rα heterodimeric Fc-fusion protein (e.g. XmAb24306) is administered at a dose selected from the group consisting of 0.01 mg/kg, 0.02 mg/kg, 0.04 mg/kg, 0.06 mg/kg, 0.09 mg/kg, and 0.12 mg/kg body weight. In some embodiments, the IL15-IL15Rα heterodimeric Fc-fusion protein (e.g., XmAb24306) is administered by IV infusion according to these dosages.

Optionally, the IL15-IL15Rα heterodimeric Fc-fusion protein is XmAb24306. In some embodiments, the dosage of XmAb24306 is about 0.0025 mg/kg body weight. In some embodiments, the dosage of XmAb24306 is about 0.01 mg/kg body weight. In some embodiments, the dosage of XmAb24306 is about 0.015 mg/kg body weight. In some embodiments, the dosage of XmAb24306 is about 0.02 mg/kg body weight. In some embodiments, the dosage of XmAb24306 is about 0.03 mg/kg body weight. In some embodiments, the dosage of XmAb24306 is about 0.04 mg/kg body weight. In some embodiments, the dosage of XmAb24306 is about 0.06 mg/kg body weight. In some embodiments, the dosage of XmAb24306 is about 0.08 mg/kg body weight. In some embodiments, the dosage of XmAb24306 is about 0.09 mg/kg body weight. In some embodiments, the dosage of XmAb24306 is about 0.12 mg/kg body weight. In some embodiments, the dosage of XmAb24306 is about 0.135 mg/kg body weight. In some embodiments, the dosage of XmAb24306 is about 0.16 mg/kg body weight. In some embodiments, the dosage of XmAb24306 is about 0.2025 mg/kg body weight. In some embodiments, the dosage of XmAb24306 is about 0.24 mg/kg body weight. In some embodiments, the dosage of XmAb24306 is about 0.32 mg/kg body weight. In some embodiments, XmAb24306 is administered at a dose selected from the group consisting of about 0.01 mg/kg, about 0.02 mg/kg, about 0.04 mg/kg, about 0.06 mg/kg, about 0.09 mg/kg, about 0.135 mg/kg, and about 0.2025 mg/kg body weight. In some embodiments, XmAb24306 is administered at a dose selected from the group consisting of about 0.01 mg/kg, about 0.02 mg/kg, about 0.04 mg/kg, about 0.06 mg/kg, about 0.09 mg/kg, and about 0.12 mg/kg body weight. In some embodiments, XmAb24306 is administered by IV infusion according to these dosages.

In some embodiments, the dosage of XmAb24306 is 0.0025 mg/kg body weight. In some embodiments, the dosage of XmAb24306 is 0.01 mg/kg body weight. In some embodiments, the dosage of XmAb24306 is 0.015 mg/kg body weight. In some embodiments, the dosage of XmAb24306 is 0.02 mg/kg body weight. In some embodiments, the dosage of XmAb24306 is 0.03 mg/kg body weight. In some embodiments, the dosage of XmAb24306 is 0.04 mg/kg body weight. In some embodiments, the dosage of XmAb24306 is 0.06 mg/kg body weight. In some embodiments, the dosage of XmAb24306 is 0.08 mg/kg body weight. In some embodiments, the dosage of XmAb24306 is 0.09 mg/kg body weight. In some embodiments, the dosage of XmAb24306 is 0.12 mg/kg body weight. In some embodiments, the dosage of XmAb24306 is 0.135 mg/kg body weight. In some embodiments, the dosage of XmAb24306 is 0.16 mg/kg body weight. In some embodiments, the dosage of XmAb24306 is 0.2025 mg/kg body weight. In some embodiments, the dosage of XmAb24306 is 0.24 mg/kg body weight. In some embodiments, the dosage of XmAb24306 is 0.32 mg/kg body weight. In some embodiments, XmAb24306 is administered at a dose selected from the group consisting of 0.01 mg/kg, 0.02 mg/kg, 0.04 mg/kg, 0.06 mg/kg, 0.09 mg/kg, 0.135 mg/kg, and 0.2025 mg/kg body weight. In some embodiments, XmAb24306 is administered at a dose selected from the group consisting of 0.01 mg/kg, 0.02 mg/kg, 0.04 mg/kg, 0.06 mg/kg, 0.09 mg/kg, and 0.12 mg/kg body weight. In some embodiments, XmAb24306 is administered by IV infusion according to these dosages.

In some embodiments, the IL15-IL15Rα heterodimeric Fc-fusion protein (e.g., XmAb24306) is administered daily, i.e., every 24 hours. In some embodiments, the IL15-IL15Rα heterodimeric Fc-fusion protein (e.g., XmAb24306) is administered weekly, i.e., once per week (Q1W). In some embodiments, the IL15-IL15Rα heterodimeric Fc-fusion protein (e.g., XmAb24306) is administered once every two weeks, i.e., once every 14 days (Q2W). In some embodiments, the IL15-IL15Rα heterodimeric Fc-fusion protein (e.g., XmAb24306) is administered once every three weeks, i.e., once every 21 days (Q3W). In some embodiments, the IL15-IL15Rα heterodimeric Fc-fusion protein (e.g., XmAb24306) is administered once every four weeks, i.e., once every 28 days (Q4W). In some embodiments, the IL15-IL15Rα heterodimeric Fc-fusion protein (e.g., XmAb24306) is administered once every five weeks (Q5W). In some embodiments, the IL15-IL15Rα heterodimeric Fc-fusion protein (e.g., XmAb24306) is administered once every six weeks (Q6W). In some embodiments, the IL15-IL15Rα heterodimeric Fc-fusion protein (e.g., XmAb24306) is administered once every month. In some embodiments, the IL15-IL15Rα heterodimeric Fc-fusion protein (e.g., XmAb24306) of the disclosure is administered by IV infusion according to the frequency disclosed herein.

In some embodiments, the IL15-IL15Rα heterodimeric Fc-fusion protein (e.g., XmAb24306) is administered at any of the above frequencies in one or more cycles. In some embodiments, the IL15-IL15Rα heterodimeric Fc-fusion protein (e.g., XmAb24306) is administered at any of the above frequencies in 1, 2, 3, 4, 5, 6, 7, 8, 9, 10, 11, 12, 13, 14, 15, 16, 17, 18, 19 or 20 cycles. In some embodiments, the IL15-IL15Rα heterodimeric Fc-fusion protein is administered at a frequency of Q1W in one or more cycles. In some embodiments, the IL15-IL15Rα heterodimeric Fc-fusion protein (e.g., XmAb24306) is administered at a frequency of Q1W in 1, 2, 3, 4, 5, 6, 7, 8, 9, 10, 11, 12, 13, 14, 15, 16, 17, 18, 19 or 20 cycles. In some embodiments, the IL15-IL15Rα heterodimeric Fc-fusion protein (e.g., XmAb24306) is administered at a frequency of Q2W in one or more cycles. In some embodiments, the IL15-IL15Rα heterodimeric Fc-fusion protein (e.g., XmAb24306) is administered at a frequency of Q2W in 1, 2, 3, 4, 5, 6, 7, 8, 9, 10, 11, 12, 13, 14, 15, 16, 17, 18, 19 or 20 cycles. In some embodiments, the IL15-IL15Rα heterodimeric Fc-fusion protein (e.g., XmAb24306) is administered at a frequency of Q3W in one or more cycles. In some embodiments, the IL15-IL15Rα heterodimeric Fc-fusion protein (e.g., XmAb24306) is administered at a frequency of Q3W in 1, 2, 3, 4, 5, 6, 7, 8, 9, 10, 11, 12, 13, 14, 15, 16, 17, 18, 19 or 20 cycles. In some embodiments, the IL15-IL15Rα heterodimeric Fc-fusion protein (e.g., XmAb24306) is administered at a frequency of Q4W in one or more cycles. In some embodiments, the IL15-IL15Rα heterodimeric Fc-fusion protein (e.g., XmAb24306) is administered at a frequency of Q4W in 1, 2, 3, 4, 5, 6, 7, 8, 9, 10, 11, 12, 13, 14, 15, 16, 17, 18, 19 or 20 cycles.

An IL15-IL15Rα heterodimeric Fc-fusion protein disclosed herein (e.g., XmAb24306) may be administered Q4W. In some embodiments, the first dose of the IL15-IL15Rα heterodimeric Fc-fusion protein (e.g., XmAb24306) is administered at least 24 hours after the first Cycle 1 dose of the FcRH5xCD3 bispecific antibody (e.g., cevostamab) or a fragment thereof that binds both antigens. In some embodiments, the first dose of the IL15-IL15Rα heterodimeric Fc-fusion protein (e.g., XmAb24306) is administered is administered between Days 2 and 4 of Cycle 1. In some embodiments, the IL15-IL15Rα heterodimeric Fc-fusion protein (e.g., XmAb24306) is administered on Day 1 of Cycle 2 and subsequent cycles.

A fourth aspect of the present disclosure provides a method of treating multiple myeloma in a subject in need thereof, the method comprising administering to the subject a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30, wherein XmAb24306 is administered intravenously at a dose from about 0.02 mg/kg to about 0.06 mg/kg and cevostamab is administered intravenously at a dose of about 132 mg.

A fifth aspect of the present disclosure provides use of a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30 in the manufacture of one or more medicaments for treating multiple myeloma in a subject in need thereof, wherein XmAb24306 is administered intravenously at a dose from about 0.02 mg/kg to about 0.06 mg/kg and cevostamab is administered intravenously at a dose of about 132 mg.

A sixth aspect of the present disclosure provides a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30 for use in treating multiple myeloma in a subject in need thereof, wherein XmAb24306 is administered intravenously at a dose from about 0.02 mg/kg to about 0.06 mg/kg and cevostamab is administered intravenously at a dose of about 132 mg.

In some embodiments, the subject has been previously administered one or more priming doses of cevostamab at a total dose of about 3.6 mg, wherein the priming dose is administered as a single dose. In some embodiments, the subject has been previously administered one or more priming doses of cevostamab at a total dose of about 3.6 mg, wherein the priming dose is administered as two doses. In some embodiments, the first priming dose is about 0.3 mg and the second priming dose is about 3.3 mg. In some embodiments, the first priming dose is administered on Day 1 and wherein the second priming dose is administered on Day 2, 3 or 4.

In some embodiments, XmAb24306 is administered at a frequency of Q4W for one or more cycles.

In some embodiments, cevostamab is administered at a frequency of Q2W for one or more cycles. In some embodiments, cevostamab is administered at a frequency of Q4W for one or more cycles.

In some embodiments, XmAb24306 is administered intravenously at a frequency of Q4W for at least seven four-week cycles, and wherein a first priming dose of 0.3 mg of cevostamab is administered intravenously on pre-phase Day 1, a second priming dose of 3.3 mg of cevostamab is administered intravenously between pre-phase Days 2-4, wherein the pre-phase is seven days, and, thereafter, at a dose of 132 mg of cevostamab is administered intravenously on Days 1 and 15 for six four-week cycles and on Day 1 for the seventh and any subsequent four-week cycles.

A seventh aspect of the present disclosure provides a method of treating multiple myeloma in a subject in need thereof, the method comprising administering to the subject a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30, wherein XmAb24306 is administered intravenously at a dose from about 0.02 mg/kg to about 0.06 mg/kg at a frequency of Q4W for at least seven four-week cycles, and wherein a first priming dose of 0.3 mg of cevostamab is administered intravenously on pre-phase Day 1, a second priming dose of 3.3 mg of cevostamab is administered intravenously between pre-phase Days 2-4, wherein the pre-phase is seven days, and, thereafter, at a dose of 132 mg of cevostamab is administered intravenously on Days 1 and 15 for six four-week cycles and on Day 1 for the seventh and any subsequent four-week cycles.

An eighth aspect of the present disclosure provides use of a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30 in the manufacture of one or more medicaments for treating multiple myeloma in a subject in need thereof, wherein XmAb24306 is administered intravenously at a dose from about 0.02 mg/kg to about 0.06 mg/kg at a frequency of Q4W for at least seven four-week cycles, and wherein a first priming dose of 0.3 mg of cevostamab is administered intravenously on pre-phase Day 1, a second priming dose of 3.3 mg of cevostamab is administered intravenously between pre-phase Days 2-4, wherein the pre-phase is seven days, and, thereafter, at a dose of 132 mg of cevostamab is administered intravenously on Days 1 and 15 for six four-week cycles and on Day 1 for the seventh and any subsequent four-week cycles.

A ninth aspect of the present disclosure provides a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30 for use in treating multiple myeloma in a subject in need thereof, wherein XmAb24306 is administered intravenously at a dose from about 0.02 mg/kg to about 0.06 mg/kg at a frequency of Q4W for at least seven four-week cycles, and wherein a first priming dose of 0.3 mg of cevostamab is administered intravenously on pre-phase Day 1, a second priming dose of 3.3 mg of cevostamab is administered intravenously between pre-phase Days 2-4, wherein the pre-phase is seven days, and, thereafter, at a dose of 132 mg of cevostamab is administered intravenously on Days 1 and 15 for six four-week cycles and on Day 1 for the seventh and any subsequent four-week cycles.

In any of the above methods, the IL15-IL15Rα heterodimeric Fc-fusion protein disclosed herein (e.g., XmAb24306) may be administered simultaneously or sequentially with the FcRH5xCD3 bispecific antibody (e.g., cevostamab) or a fragment thereof that binds both antigens. In some embodiments of any of the above methods, the IL15-IL15Rα heterodimeric Fc-fusion protein disclosed herein (e.g., XmAb24306) and an FcRH5xCD3 bispecific antibody (e.g., cevostamab) or a fragment thereof that binds both antigens are administered simultaneously. In some embodiments of any of the above methods, the IL15-IL15Rα heterodimeric Fc-fusion protein disclosed herein (e.g., XmAb24306) and an FcRH5xCD3 bispecific antibody (e.g., cevostamab) or a fragment thereof that binds both antigens are administered sequentially. In some embodiments of any of the above methods, the FcRH5xCD3 bispecific antibody (e.g., cevostamab) or a fragment thereof that binds both antigens is administered after administering the IL15-IL15Rα heterodimeric Fc-fusion protein disclosed herein (e.g., XmAb24306). In some embodiments of any of the above methods, the FcRH5xCD3 bispecific antibody (e.g. cevostamab) or a fragment thereof that binds both antigens is administered before administering the IL15-IL15Rα heterodimeric Fc-fusion protein disclosed herein (e.g., XmAb24306). In some embodiments of any of the above methods, the IL15-IL15Rα heterodimeric Fc-fusion protein disclosed herein (e.g., XmAb24306) and the FcRH5xCD3 bispecific antibody (e.g., cevostamab) or a fragment thereof that binds both antigens are administered in the same composition. In some embodiments of any of the above methods, the IL15-IL15Rα heterodimeric Fc-fusion protein disclosed herein (e.g., XmAb24306) are administered in a different composition than the FcRH5xCD3 bispecific antibody (e.g., cevostamab) or a fragment thereof that binds both antigens.

In some embodiments of any of the above methods, the IL15-IL15Rα heterodimeric Fc-fusion protein disclosed herein (e.g., XmAb24306) and the FcRH5xCD3 bispecific antibody (e.g., cevostamab) or a fragment thereof that binds both antigens may synergize. In some embodiments of any of the above methods, the IL15-IL15Rα heterodimeric Fc-fusion protein (e.g., XmAb24306) may be administered at a dosage less than its therapeutically effective dose when administered as a monotherapy. In some embodiments of any of the above methods, the FcRH5xCD3 bispecific antibody (e.g., cevostamab) or a fragment thereof that binds both antigens may be administered at a dosage less than its therapeutically effective dose when administered as a monotherapy.

In some embodiments of any of the above methods, the method further comprises administering a corticosteroid to the subject. In some embodiments, the corticosteroid is administered to a subject with signs or symptoms of CRS. CRS symptoms can be progressive, must include fever at the onset, and may include hypotension, capillary leak (hypoxia) and end organ dysfunction. The fever should cause a temperature ≥38° C. (100.4° F.) and may be prolonged (e.g., greater than 4 hours, such as greater than 6 hours). In some embodiments, the corticosteroid is administered intravenously. In some embodiments, the corticosteroid is dexamethasone. In some embodiments, the dexamethasone is administered at a dose of 10 mg. In some embodiments, the 10 mg of dexamethasone is administered intravenously every six hours.

In some embodiments of any of the above methods, the method further comprises administering a therapeutically effective amount of tocilizumab. Tocilizumab (Actemra®/RoActemra®) is a recombinant, humanized, anti-human monoclonal antibody directed against soluble and membrane-bound IL 6R, which inhibits IL-6 mediated signaling. Emerging evidence implicates IL-6 as a central mediator in CRS. Without being bound by theory, blocking the inflammatory action of IL-6 using tocilizumab could therefore represent an effective approach for the treatment of CRS. Indeed, the U.S. Food and Drug Administration has approved tocilizumab for the treatment of severe or life-threatening CAR-T cell-induced CRS in adults and in pediatric patients 2 years of age and older. However recent literature supports use of tocilizumab in all grades of CRS (Neelapu et al. 2018; Riegler et al. 2019). Accordingly, patients treated with a FcRH5xCD3 bispecific antibody (e.g., cevostamab) who develop CRS may benefit from tocilizumab therapy.

In some embodiments of any of the above methods, the tocilizumab is administered to subjects who experience a CRS event. In some embodiments of any of the above methods, the tocilizumab is administered to subjects remain refractory to a corticosteroid 24 hours after the first corticosteroid dose. In some embodiments of any of the above methods, the tocilizumab is administered at a dose of 8 mg/kg. In some embodiments of any of the above methods, the tocilizumab is administered at a dose of 12 mg/kg. In some embodiments of any of the above methods, the tocilizumab is administered at a dose of 8 mg/kg if the patient's weight is ≥30 kg. In some embodiments of any of the above methods, the tocilizumab is administered at a dose of 12 mg/kg if the patient's weight is <30 kg. In some embodiments of any of the above methods, the tocilizumab is administered intravenously. In some embodiments, the tocilizumab is administered every 8 hours.

In some embodiments of any of the above methods, the subject has been previously administered an agent for the treatment of the blood cancer. In some embodiments of any of the above methods, the subject has been previously administered one or more prior treatments for treatment of the blood cancer. In some embodiments of any of the above methods, the subject has been previously administered one prior treatment. In some embodiments of any of the above methods, the subject has been previously administered two prior treatments. In some embodiments of any of the above methods, the subject has been previously administered three prior treatments. In some embodiments of any of the above methods, the subject has been previously administered four prior treatments. In some embodiments, the subject has been previously administered five prior treatments. In some embodiments of any of the above methods, the prior treatment administered to the subject is an immunomodulatory drug, a proteasome inhibitor, an anti-CD38 monoclonal antibody, or a combination thereof. In some embodiments of any of the above methods, the subject has previously been administered at least three prior treatments, including at least one immunomodulatory drug, proteasome inhibitor, or anti-CD38 monoclonal antibody. In some embodiments of any of the above methods, the subject has previously been administered at least three prior treatments, including at least one immunomodulatory drug, proteasome inhibitor, and anti-CD38 monoclonal antibody. In some embodiments, the prior treatment administered to the subject is an immunomodulatory drug. In some embodiments of any of the above methods, the immunomodulatory drug is selected from the group consisting of lenalidomide, thalidomide, and pomalidomide. In some embodiments of any of the above methods, the immunomodulatory drug is lenalidomide. In some embodiments of any of the above methods, the immunomodulatory drug is thalidomide. In some embodiments of any of the above methods, the immunomodulatory drug is pomalidomide. In some embodiments of any of the above methods, the prior treatment administered to the subject is a proteasome inhibitor. In some embodiments of any of the above methods, the proteasome inhibitor is selected from the group consisting of bortezomib, carfilzomib, and ixazomib. In some embodiments of any of the above methods, the proteasome inhibitor is selected from the group consisting of bortezomib. In some embodiments of any of the above methods, the proteasome inhibitor is selected from the group consisting of carfilzomib. In some embodiments of any of the above methods, the proteasome inhibitor is selected from the group consisting of ixazomib. In some embodiments of any of the above methods, the prior treatment administered to the subject is an anti-CD38 monoclonal antibody. In some embodiments of any of the above methods, the anti-CD38 monoclonal antibody is selected from the group consisting of daratumumab, isatuximab, mezagitamab (TAK-079) and felzartamab (MOR202). In some embodiments of any of the above methods, the anti-CD38 monoclonal antibody is daratumumab. In some embodiments of any of the above methods, the anti-CD38 monoclonal antibody is isatuximab. In some embodiments of any of the above methods, the anti-CD38 monoclonal antibody is mezagitamab. In some embodiments of any of the above methods, the anti-CD38 monoclonal antibody is felzartamab.

### Exemplary Embodiments

Particular embodiments of the disclosure are set forth in the following numbered paragraphs:
1. A method of treating a blood cancer in a subject in need thereof, the method comprising administering to the subject a therapeutically effective amount of (a) an IL15-IL15Rα heterodimeric Fc-fusion protein and (b) an FcRH5xCD3 bispecific antibody or a fragment thereof that binds both antigens.
2. Use of a therapeutically effective amount of (a) an IL15-IL15Rα heterodimeric Fc-fusion protein and (b) an FcRH5xCD3 bispecific antibody or a fragment thereof that binds both antigens in the manufacture of one or more medicaments for the treatment of a blood cancer in a subject in need thereof.
3. A therapeutically effective amount of (a) an IL15-IL15Rα heterodimeric Fc-fusion protein and (b) an FcRH5xCD3 bispecific antibody or a fragment thereof that binds both antigen for use in treating a blood cancer in a subject in need thereof.
4. The method according to embodiment 1, the use of embodiment 2, or the IL15-IL15Rα heterodimeric Fc-fusion protein and FcRH5xCD3 bispecific antibody or a fragment thereof of embodiment 3, wherein the IL15-IL15Rα heterodimeric Fc-fusion protein comprises: (a) a first fusion protein comprising an interleukin-15 (IL-15) protein covalently attached to the N-terminus of a first Fc domain via a first domain linker, wherein the IL-15 protein comprises the amino acid sequence of SEQ ID NO: 5 and wherein the first Fc domain is a variant of a human IgG1 Fc domain; and (b) a second fusion protein comprising an IL-15 receptor alpha (IL-15Rα) protein fragment covalently attached to the N-terminus of a second Fc domain via a second domain linker, wherein the IL-15Rα protein comprises the amino acid sequence of SEQ ID NO: 4 and wherein the second Fc domain is a variant of a human IgG1 Fc domain.
5. The method, the use, or the IL15-IL15Rα heterodimeric Fc-fusion protein and FcRH5xCD3 bispecific antibody or a fragment thereof according to embodiment 4, wherein the IL15-IL15Rα heterodimeric Fc-fusion protein comprises: (a) a first fusion protein comprising an interleukin-15 (IL-15) protein covalently attached to the N-terminus of a first Fc domain via a first domain linker, wherein the IL-15 protein comprises the amino acid sequence of SEQ ID NO: 5 and wherein the first Fc domain comprises the amino acid sequence of SEQ ID NO: 6; and (b) a second fusion protein comprising an IL-15 receptor alpha (IL-15Rα) protein fragment covalently attached to the N-terminus of a second Fc domain via a second domain linker, wherein the IL-15Rα protein comprises the amino acid sequence of SEQ ID NO: 4 and wherein the second Fc domain comprises the amino acid sequence of SEQ ID NO: 7.
6. The method according to any one of embodiments 4-5, the use of any one of embodiments 4-5, or the IL15-IL15Rα heterodimeric Fc-fusion protein and FcRH5xCD3 bispecific antibody or a fragment thereof of any one of embodiments 4-5, wherein the first domain linker comprises the amino acid sequence of SEQ ID NO: 8.
7. The method according to any one of embodiments 4-6, the use of any one of embodiments 4-6, or the IL15-IL15Rα heterodimeric Fc-fusion protein and FcRH5xCD3 bispecific antibody or a fragment thereof of any one of embodiments 4-6, wherein the second domain linker comprises the amino acid sequence of SEQ ID NO: 8.
8. The method according to any one of embodiments 4-7, the use of any one of embodiments 4-7, or the IL15-IL15Rα heterodimeric Fc-fusion protein and FcRH5xCD3 bispecific antibody or a fragment thereof of any one of embodiments 4-7, wherein the first domain linker comprises the amino acid sequence of SEQ ID NO: 8 and the second domain linker comprises the amino acid sequence of SEQ ID NO: 8.
9. The method according to any one of embodiments 4-8, the use of any one of embodiments 4-8, or the IL15-IL15Rα heterodimeric Fc-fusion protein and FcRH5xCD3 bispecific antibody or a fragment thereof of any one of embodiments 4-8, wherein the first fusion protein comprises the amino acid sequence of SEQ ID NO: 9.
10. The method according to any one of embodiments 4-9, the use of any one of embodiments 4-9, or the IL15-IL15Rα heterodimeric Fc-fusion protein and FcRH5xCD3 bispecific antibody or a fragment thereof of any one of embodiments 4-9, wherein the second fusion protein comprises the amino acid sequence of SEQ ID NO: 10.
11. The method according to any one of embodiments 1 and 4-10, the use of any one of embodiments 2 and 4-10, or the IL15-IL15Rα heterodimeric Fc-fusion protein and FcRH5xCD3 bispecific antibody or a fragment thereof of any one of embodiments 3-10, wherein the heterodimeric protein comprises a first fusion protein comprising the amino acid sequence set forth in SEQ ID NO: 9, and a second fusion protein comprising the amino acid sequence set forth in SEQ ID NO: 10.
12. The method according to any one of embodiments 1 and 4-11, the use of any one of embodiments 2 and 4-11, or the IL15-IL15Rα heterodimeric Fc-fusion protein and FcRH5xCD3 bispecific antibody or a fragment thereof of any one of embodiments 3-11, wherein the IL15-IL15Rα heterodimeric Fc-fusion protein is XmAb24306.
13. The method according to any one of embodiments 1 and 4-12, the use according to any one of embodiments 2 and 4-12, or the IL15-IL15Rα heterodimeric Fc-fusion protein and FcRH5xCD3 bispecific antibody or a fragment thereof according to any one of embodiments 3-12, wherein the FcRH5xCD3 bispecific antibody or a fragment thereof that binds both antigens comprises an anti-FcRH5 light chain variable region, an anti-FcRH5 heavy chain variable region, an anti-CD3 light chain variable region, and an anti-CD3 heavy chain variable region.
14. The method, the use, or the IL15-IL15Rα heterodimeric Fc-fusion protein and FcRH5xCD3 bispecific antibody or a fragment thereof according to embodiment 13, wherein the anti-FcRH5 light chain variable region comprises a light chain complementarity determining region-1 (CDR-L1) comprising the amino acid sequence of SEQ ID NO: 11, a CDR-L2 comprising the amino acid sequence of SEQ ID NO: 12, and a CDR-L3 comprising the amino acid sequence of SEQ ID NO: 13; and the anti-FcRH5 heavy chain variable region comprises a heavy chain complementarity determining region-1 (CDR-H1) comprising the amino acid sequence of SEQ ID NO: 14, a CDR-H2 comprising the amino acid sequence of SEQ ID NO: 15, and a CDR-H3 comprising the amino acid sequence of SEQ ID NO: 16.
15. The method, the use, or the IL15-IL15Rα heterodimeric Fc-fusion protein and FcRH5xCD3 bispecific antibody or a fragment thereof according to embodiment 13 or 14, wherein the anti-FcRH5 light chain variable region comprises the amino acid sequence of SEQ ID NO: 17.
16. The method, the use, or the IL15-IL15Rα heterodimeric Fc-fusion protein and FcRH5xCD3 bispecific antibody or a fragment thereof according to any one of embodiments 13-15, wherein the anti-FcRH5 heavy chain variable region comprises the amino acid sequence of SEQ ID NO: 18.
17. The method, the use, or the IL15-IL15Rα heterodimeric Fc-fusion protein and FcRH5xCD3 bispecific antibody or a fragment thereof according to any one of embodiments 13-16, wherein the anti-FcRH5 light chain comprises the amino acid sequence of SEQ ID NO: 19.
18. The method, the use, or the IL15-IL15Rα heterodimeric Fc-fusion protein and FcRH5xCD3 bispecific antibody or a fragment thereof according to any one of embodiments 13-17, wherein the anti-FcRH5 heavy chain comprises the amino acid sequence of SEQ ID NO: 20.
19. The method, the use, or the IL15-IL15Rα heterodimeric Fc-fusion protein and FcRH5xCD3 bispecific antibody or a fragment thereof according to any one of embodiments 13-18, wherein the anti-CD3 light chain variable region comprises a light chain complementarity determining region-1 (CDR-L1) comprising the amino acid sequence of SEQ ID NO: 21, a CDR-L2 comprising the amino acid sequence of SEQ ID NO: 22, and a CDR-L3 comprising the amino acid sequence of SEQ ID NO: 23; and the anti-CD3 heavy chain variable region comprises a heavy chain complementarity determining region-1 (CDR-H1) comprising the amino acid sequence of SEQ ID NO: 24, a CDR-H2 comprising the amino acid sequence of SEQ ID NO: 25, and a CDR-H3 comprising the amino acid sequence of SEQ ID NO: 26.
20. The method, the use, or the IL15-IL15Rα heterodimeric Fc-fusion protein and FcRH5xCD3 bispecific antibody or a fragment thereof according to embodiment 19, wherein the anti-CD3 light chain variable region comprises the amino acid sequence of SEQ ID NO: 27.
21. The method, the use, or the IL15-IL15Rα heterodimeric Fc-fusion protein and FcRH5xCD3 bispecific antibody or a fragment thereof according to embodiment 19 or 20, wherein the anti-CD3 heavy chain variable region comprises the amino acid sequence of SEQ ID NO: 28.
22. The method, the use, or the IL15-IL15Rα heterodimeric Fc-fusion protein and FcRH5xCD3 bispecific antibody or a fragment thereof according to any one of embodiments 19-21, wherein the anti-CD3 light chain comprises the amino acid sequence of SEQ ID NO: 29.
23. The method, the use, or the IL15-IL15Rα heterodimeric Fc-fusion protein and FcRH5xCD3 bispecific antibody or a fragment thereof according to any one of embodiments 19-22, wherein the anti-CD3 heavy chain comprises the amino acid sequence of SEQ ID NO: 30.
24. The method, the use, or the IL15-IL15Rα heterodimeric Fc-fusion protein and FcRH5xCD3 bispecific antibody or a fragment thereof according to any one of embodiments 19-23, wherein the anti-FcRH5 light chain variable region comprises the amino acid sequence of SEQ ID NO: 17; the anti-FcRH5 heavy chain variable region comprises the amino acid sequence of SEQ ID NO: 18; the anti-CD3 light chain variable region comprises the amino acid sequence of SEQ ID NO: 27; and the anti-CD3 heavy chain variable region comprises the amino acid sequence of SEQ ID NO: 28.
25. The method, the use, or the IL15-IL15Rα heterodimeric Fc-fusion protein and FcRH5xCD3 bispecific antibody or a fragment thereof according to any one of embodiments 19-24, wherein the anti-FcRH5 light chain comprises the amino acid sequence of SEQ ID NO: 19; the anti-FcRH5 heavy chain comprises the amino acid sequence of SEQ ID NO: 20; the anti-CD3 light chain comprises the amino acid sequence of SEQ ID NO: 29; and the anti-CD3 heavy chain comprises the amino acid sequence of SEQ ID NO: 30.
26. The method, the use, or the IL15-IL15Rα heterodimeric Fc-fusion protein and FcRH5xCD3 bispecific antibody or a fragment thereof according to any one of embodiments 19-25, wherein the FcRH5xCD3 bispecific antibody is cevostamab.
27. The method according to any one of embodiments 1 and 4-26, the use according to any one of embodiments 2 and 4-26, or the IL15-IL15Rα heterodimeric Fc-fusion protein and FcRH5xCD3 bispecific antibody or a fragment thereof according to any one of embodiments 3-26, wherein said blood cancer is selected from the group consisting of leukemia, acute myeloid leukemia, adult acute lymphoblastic leukemia, chronic lymphocytic leukemia, lymphoma, non-Hodgkin's lymphoma, B-cell non-Hodgkin's lymphoma, and multiple myeloma.
28. The method, the use, or the IL15-IL15Rα heterodimeric Fc-fusion protein and FcRH5xCD3 bispecific antibody or a fragment thereof according to embodiment 27, wherein said blood cancer is multiple myeloma.
29. The method, the use, or the IL15-IL15Rα heterodimeric Fc-fusion protein and FcRH5xCD3 bispecific antibody or a fragment thereof according to embodiment 28, wherein said multiple myeloma is relapsed or refractory multiple myeloma.
30. The method according to any one of embodiments 1 and 4-29, the use according to any one of embodiments 2 and 4-29, or the IL15-IL15Rα heterodimeric Fc-fusion protein and FcRH5xCD3 bispecific antibody or a fragment thereof according to any one of embodiments 3-29, wherein the subject has been previously administered one or more prior treatments.
31. The method, the use, or the IL15-IL15Rα heterodimeric Fc-fusion protein and FcRH5xCD3 bispecific antibody or a fragment thereof according to embodiment 30, wherein the prior treatment is an immunomodulatory drug, a proteasome inhibitor, or an anti-CD38 monoclonal antibody.
32. The method, the use, or the IL15-IL15Rα heterodimeric Fc-fusion protein and FcRH5xCD3 bispecific antibody or a fragment thereof according to embodiment 31, wherein the immunomodulatory drug is selected from the group consisting of lenalidomide, thalidomide, and pomalidomide.
33. The method, the use, or the IL15-IL15Rα heterodimeric Fc-fusion protein and FcRH5xCD3 bispecific antibody or a fragment thereof according to embodiment 31, wherein the proteasome inhibitor is selected from the group consisting of bortezomib, carfilzomib, and ixazomib.
34. The method, the use, or the IL15-IL15Rα heterodimeric Fc-fusion protein and FcRH5xCD3 bispecific antibody or a fragment thereof according to embodiment 31, wherein the anti-CD38 monoclonal antibody is selected from the group consisting of daratumumab, isatuximab, mezagitamab, and felzartamab.
35. The method according to any one of embodiments 1 and 4-33, the use according to any one of embodiments 2 and 4-34, or the IL15-IL15Rα heterodimeric Fc-fusion protein and FcRH5xCD3 bispecific antibody or a fragment thereof according to any one of embodiments 3-34, wherein the IL15-IL15Rα heterodimeric Fc-fusion protein is administered at a dose selected from the group consisting of about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.1 mg/kg, about 0.12 mg/kg, about 0.16 mg/kg, about 0.2 mg/kg, about 0.24 mg/kg and about 0.32 mg/kg body weight.
36. The method, the use, or the IL15-IL15Rα heterodimeric Fc-fusion protein and FcRH5xCD3 bispecific antibody or a fragment thereof according to embodiment 35, wherein the IL15-IL15Rα heterodimeric Fc-fusion protein is administered at a dose selected from the group consisting of about 0.01 mg/kg, about 0.02 mg/kg, about 0.04 mg/kg, about 0.06 mg/kg, about 0.09 mg/kg and about 0.12 mg/kg body weight.
37. The method according to any one of embodiments 1 and 4-36, the use according to any one of embodiments 2 and 4-36, or the IL15-IL15Rα heterodimeric Fc-fusion protein and FcRH5xCD3 bispecific antibody or a fragment thereof according to any one of embodiments 3-36, wherein the IL15-IL15Rα heterodimeric Fc-fusion protein is administered at a dose selected from the group consisting of 0.0025 mg/kg, 0.005 mg/kg, 0.01 mg/kg, 0.015 mg/kg, 0.02 mg/kg, 0.025 mg/kg, 0.03 mg/kg, 0.04 mg/kg, 0.05 mg/kg, 0.06 mg/kg, 0.08 mg/kg, 0.10 mg/kg, 0.16 mg/kg, 0.20 mg/kg, 0.24 mg/kg and 0.32 mg/kg body weight.
38. The method, the use, or the IL15-IL15Rα heterodimeric Fc-fusion protein and FcRH5xCD3 bispecific antibody or a fragment thereof according to embodiment 36, wherein the IL15-IL15Rα heterodimeric Fc-fusion protein is administered at a dose selected from the group consisting of 0.01 mg/kg, 0.02 mg/kg, 0.04 mg/kg, 0.06 mg/kg, 0.09 mg/kg and 0.12 mg/kg body weight.
39. The method according to any one of embodiments 1 and 4-38, the use according to any one of embodiments 2 and 4-38, or the IL15-IL15Rα heterodimeric Fc-fusion protein and FcRH5xCD3 bispecific antibody or a fragment thereof according to any one of embodiments 3-38, wherein the IL15-IL15Rα heterodimeric Fc-fusion protein is administered at a frequency selected from the group consisting of Q1W, Q2W, Q3W, Q4W, Q5W and Q6W.
40. The method, the use, or the IL15-IL15Rα heterodimeric Fc-fusion protein and FcRH5xCD3 bispecific antibody or a fragment thereof according to embodiment 39, wherein the IL15-IL15Rα heterodimeric Fc-fusion protein is administered at a frequency of Q1W in one or more cycles.
41. The method, the use, or the IL15-IL15Rα heterodimeric Fc-fusion protein and FcRH5xCD3 bispecific antibody or a fragment thereof according to embodiment 39, wherein the IL15-IL15Rα heterodimeric Fc-fusion protein is administered at a frequency of Q2W in one or more cycles.
42. The method, the use, or the IL15-IL15Rα heterodimeric Fc-fusion protein and FcRH5xCD3 bispecific antibody or a fragment thereof according to embodiment 39, wherein the IL15-IL15Rα heterodimeric Fc-fusion protein is administered at a frequency of Q4W in one or more cycles.
43. The method according to any one of embodiments 1 and 4-42, the use according to any one of embodiments 2 and 4-42, or the IL15-IL15Rα heterodimeric Fc-fusion protein and FcRH5xCD3 bispecific antibody or a fragment thereof according to any one of embodiments 3-42, wherein the IL15-IL15Rα heterodimeric Fc-fusion protein is administered intravenously.
44. The method according to any one of embodiments 1 and 4-43, the use according to any one of embodiments 2 and 4-43, or the IL15-IL15Rα heterodimeric Fc-fusion protein and FcRH5xCD3 bispecific antibody or a fragment thereof according to any one of embodiments 3-43, wherein the IL15-IL15Rα heterodimeric Fc-fusion protein and said FcRH5xCD3 bispecific antibody or fragment thereof that binds both antigens are administered simultaneously.
45. The method according to any one of embodiments 1 and 4-43, the use according to any one of embodiments 2 and 4-43, or the IL15-IL15Rα heterodimeric Fc-fusion protein and FcRH5xCD3 bispecific antibody or a fragment thereof according to any one of embodiments 3-43, wherein the IL15-IL15Rα heterodimeric Fc-fusion protein and said FcRH5xCD3 bispecific antibody or fragment thereof that binds both antigens are administered sequentially.
46. The method according to any one of embodiments 1 and 4-45, the use according to any one of embodiments 2 and 4-43, or the IL15-IL15Rα heterodimeric Fc-fusion protein and FcRH5xCD3 bispecific antibody or a fragment thereof according to any one of embodiments 3-43, wherein said FcRH5xCD3 bispecific antibody or fragment thereof that binds both antigens is administered at a frequency selected from the group consisting of Q1W, Q2W, Q3W, Q4W, Q5W and Q6W in one or more cycles.
47. The method, the use, or the IL15-IL15Rα heterodimeric Fc-fusion protein and FcRH5xCD3 bispecific antibody or a fragment thereof according to embodiment 46, wherein said FcRH5xCD3 bispecific antibody or fragment thereof that binds both antigens is administered at a frequency of Q2W in one or more cycles.
48. The method, the use, or the IL15-IL15Rα heterodimeric Fc-fusion protein and FcRH5xCD3 bispecific antibody or a fragment thereof according to embodiment 47, wherein said IL15-IL15Rα heterodimeric Fc-fusion protein is administered at a frequency of Q4W, and wherein said FcRH5xCD3 bispecific antibody or fragment thereof that binds both antigens is administered at a frequency of Q2W in one or more cycles.
49. The method, the use, or the IL15-IL15Rα heterodimeric Fc-fusion protein and FcRH5xCD3 bispecific antibody or a fragment thereof according to embodiment 46, wherein said FcRH5xCD3 bispecific antibody or fragment thereof that binds both antigens is administered at a frequency of Q4W in one or more cycles.
50. The method, the use, or the IL15-IL15Rα heterodimeric Fc-fusion protein and FcRH5xCD3 bispecific antibody or a fragment thereof according to any one of embodiments 40-49, wherein each of the one or more cycles is a four-week cycle.
51. The method, the use, or the IL15-IL15Rα heterodimeric Fc-fusion protein and FcRH5xCD3 bispecific antibody or a fragment thereof according to embodiment 50, wherein the FcRH5xCD3 bispecific antibody or a fragment thereof that binds both antigens is administered Q2W for six four-week cycles and administered Q4W in the seventh and any subsequent four-week cycle.
52. The method, the use, or the IL15-IL15Rα heterodimeric Fc-fusion protein and FcRH5xCD3 bispecific antibody or a fragment thereof according to embodiment 51, wherein the FcRH5xCD3 bispecific antibody or a fragment thereof that binds both antigens is administered on Days 1 and 15 for six four-week cycles and administered on Day 1 in the seventh and any subsequent four-week cycle.
53. The method according to any one of embodiments 1 and 4-52, the use according to any one of embodiments 2 and 4-52, or the IL15-IL15Rα heterodimeric Fc-fusion protein and FcRH5xCD3 bispecific antibody or a fragment thereof according to any one of embodiments 3-52, wherein the FcRH5xCD3 bispecific antibody is administered at a dose of about 132 mg to about 198 mg.
54. The method, the use, or the IL15-IL15Rα heterodimeric Fc-fusion protein and FcRH5xCD3 bispecific antibody or a fragment thereof according to embodiment 53, wherein the FcRH5xCD3 bispecific antibody is administered at a dose of about 132 mg.
55. The method, the use, or the IL15-IL15Rα heterodimeric Fc-fusion protein and FcRH5xCD3 bispecific antibody or a fragment thereof according to embodiment 54, wherein the FcRH5xCD3 bispecific antibody is administered at a dose of about 132 mg at a frequency of Q2W in one or more 28-day cycles.
56. The method, the use, or the IL15-IL15Rα heterodimeric Fc-fusion protein and FcRH5xCD3 bispecific antibody or a fragment thereof according to embodiment 53, wherein the FcRH5xCD3 bispecific antibody is administered at a dose of about 160 mg.
57. The method, the use, or the IL15-IL15Rα heterodimeric Fc-fusion protein and FcRH5xCD3 bispecific antibody or a fragment thereof according to embodiment 53, wherein the FcRH5xCD3 bispecific antibody is administered at a dose of about 198 mg.
58. The method, the use, or the IL15-IL15Rα heterodimeric Fc-fusion protein and FcRH5xCD3 bispecific antibody or a fragment thereof according to any one of embodiments 46-57, wherein one or more priming doses of the FcRH5xCD3 bispecific antibody or a fragment thereof that binds both antigens is administered to the subject during a pre-phase before the first treatment cycle.
59. The method, the use, or the IL15-IL15Rα heterodimeric Fc-fusion protein and FcRH5xCD3 bispecific antibody or a fragment thereof according to embodiment 58, wherein the pre-phase is seven days.
60. The method, the use, or the IL15-IL15Rα heterodimeric Fc-fusion protein and FcRH5xCD3 bispecific antibody or a fragment thereof according to embodiment 58 or 59, wherein the priming dose of the FcRH5xCD3 bispecific antibody is about 3.6 mg.
61. The method, the use, or the IL15-IL15Rα heterodimeric Fc-fusion protein and FcRH5xCD3 bispecific antibody or a fragment thereof according to anyone of embodiments 58-60, wherein two priming doses of the FcRH5xCD3 bispecific antibody or a fragment thereof that binds both antigens are administered to the subject.
62. The method, the use, or the IL15-IL15Rα heterodimeric Fc-fusion protein and FcRH5xCD3 bispecific antibody or a fragment thereof according to embodiment 61, wherein the first priming dose is administered on pre-phase Day 1.
63. The method, the use, or the IL15-IL15Rα heterodimeric Fc-fusion protein and FcRH5xCD3 bispecific antibody or a fragment thereof according to embodiment 62, wherein the second priming dose is administered between pre-phase Days 2-4.
64. The method, the use, or the IL15-IL15Rα heterodimeric Fc-fusion protein and FcRH5xCD3 bispecific antibody or a fragment thereof according to any one of embodiments 61-63, wherein the minimum interval between the end of the first priming dose and the initiation of the second priming dose is 20 hours.
65. The method, the use, or the IL15-IL15Rα heterodimeric Fc-fusion protein and FcRH5xCD3 bispecific antibody or a fragment thereof according to any one of embodiments 61-64, wherein about 3.6 mg of the FcRH5xCD3 bispecific antibody is administered between the two priming doses.
66. The method, the use, or the IL15-IL15Rα heterodimeric Fc-fusion protein and FcRH5xCD3 bispecific antibody or a fragment thereof according to embodiment 65, wherein the first priming dose is about 0.3 mg and the second priming dose is about 3.3 mg.
67. The method according to any one of embodiments 1 and 4-55 and 58-66, the use according to any one of embodiments 2 and 4-55 and 58-66, or the IL15-IL15Rα heterodimeric Fc-fusion protein and FcRH5xCD3 bispecific antibody or a fragment thereof according to any one of embodiments 3-55 and 58-66, wherein the subject is administered a first priming dose of about 0.3 mg of the FcRH5xCD3 bispecific antibody on pre-phase Day 1, a second priming dose of about 3.3 mg of the FcRH5xCD3 bispecific antibody between pre-phase Days 2-4 and, thereafter, at a dose of about 132 mg of the FcRH5xCD3 bispecific antibody on Days 1 and 15 for six four-week cycles and on Day 1 for the seventh and any subsequent four-week cycles.
68. The method according to any one of embodiments 1 and 4-67, the use according to any one of embodiments 2 and 4-67, or the IL15-IL15Rα heterodimeric Fc-fusion protein and FcRH5xCD3 bispecific antibody or a fragment there of according to any one of embodiments 3-67, wherein said FcRH5xCD3 bispecific antibody is administered intravenously.
69. The method according to any one of embodiments 1 and 4-68, the use according to any one of embodiments 2 and 4-68, or the IL15-IL15Rα heterodimeric Fc-fusion protein and FcRH5xCD3 bispecific antibody or a fragment thereof according to any one of embodiments 3-68, wherein the method further comprises administering to the subject a therapeutically effective amount of tocilizumab.
70. The method, the use or the IL15-IL15Rα heterodimeric Fc-fusion protein and FcRH5xCD3 bispecific antibody or a fragment thereof according to embodiment 69, wherein the subject has suffered a cytokine release syndrome (CRS) event.
71. The method, the use, or the IL15-IL15Rα heterodimeric Fc-fusion protein and FcRH5xCD3 bispecific antibody or a fragment thereof according to embodiment 69 or 70, wherein the tocilizumab is administered to subjects that remain refractory to a corticosteroid 24 hours after the first corticosteroid dose.
72. The method, the use, or the IL15-IL15Rα heterodimeric Fc-fusion protein and FcRH5xCD3 bispecific antibody or a fragment thereof according to any one of embodiments 69-71, wherein the tocilizumab is administered at a dose of 8 mg/kg.
73. The method, the use, or the IL15-IL15Rα heterodimeric Fc-fusion protein and FcRH5xCD3 bispecific antibody or a fragment thereof according to embodiment 72, wherein the tocilizumab is administered at a dose of 8 mg/kg if the subject's weight is ≥30 kg.
74. The method, the use, or the IL15-IL15Rα heterodimeric Fc-fusion protein and FcRH5xCD3 bispecific antibody or a fragment thereof according to any one of embodiments 69-71, wherein the tocilizumab is administered at a dose of 12 mg/kg.
75. The method, the use, or the IL15-IL15Rα heterodimeric Fc-fusion protein and FcRH5xCD3 bispecific antibody or a fragment thereof according to embodiment 74, wherein the tocilizumab is administered at a dose of 12 mg/kg if the patient's weight is <30 kg.
76. The method, the use, or the IL15-IL15Rα heterodimeric Fc-fusion protein and FcRH5xCD3 bispecific antibody or a fragment thereof according to any one of embodiments 69-75, wherein the tocilizumab is administered intravenously.
77. The method, the use, or the IL15-IL15Rα heterodimeric Fc-fusion protein and FcRH5xCD3 bispecific antibody or a fragment thereof according to any one of embodiments 69-76, wherein the tocilizumab is administered every 8 hours.
78. A method of treating multiple myeloma in a subject in need thereof, the method comprising administering to the subject a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30, wherein XmAb24306 is administered intravenously at a dose from about 0.02 mg/kg to about 0.06 mg/kg and cevostamab is administered intravenously at a dose of about 132 mg.
79. Use of a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30 in the manufacture of one or more medicaments for treating multiple myeloma in a subject in need thereof, wherein XmAb24306 is formulated to be administered intravenously at a dose from about 0.02 mg/kg to about 0.06 mg/kg and cevostamab is formulated to be administered intravenously at a dose of about 132 mg.
80. A therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30 for use in treating multiple myeloma in a subject in need thereof, wherein XmAb24306 is administered intravenously at a dose from about 0.02 mg/kg to about 0.06 mg/kg and cevostamab is administered intravenously at a dose of about 132 mg.
81. The method according to embodiment 78, the use according to embodiment 79, or the XmAb24306 and cevostamab according to embodiment 80, wherein the treatment further comprises administering to the subject one or more priming doses of cevostamab at a total dose of about 3.6 mg during a seven-day pre-phase before the first treatment cycle, wherein the priming dose is administered as a single dose.
82. The method according to embodiment 78, the use according to embodiment 79, or the XmAb24306 and cevostamab according to embodiment 80, wherein the treatment further comprises administering to the subject one or more priming doses of cevostamab at a total dose of about 3.6 mg during a seven-day pre-phase before the first treatment cycle, wherein the priming dose is administered as two doses.
83. The method, the use, or the XmAb24306 and cevostamab according to embodiment 82, wherein the first priming dose is about 0.3 mg and the second priming dose is about 3.3 mg.
84. The method, the use, or the XmAb24306 and cevostamab according to embodiment 83, wherein the first priming dose is administered on Day 1 of the pre-phase and wherein the second priming dose is administered on Day 2, 3 or 4 of the pre-phase.
85. The method according to any one of embodiments 78 and 81-84, the use according to any one of embodiments 79 and 81-84, or the XmAb24306 and cevostamab according to any one of embodiments 80-84, wherein XmAb24306 is administered at a frequency of Q4W for one or more cycles.
86. The method according to any one of embodiments 78 and 81-85, the use according to any one of embodiments 79 and 81-85, or the XmAb24306 and cevostamab according to any one of embodiments 80-85, wherein cevostamab is administered at a frequency of Q2W for one or more cycles.
87. The method according to any one of embodiments 78 and 81-86, the use according to any one of embodiments 79 and 81-86, or the XmAb24306 and cevostamab according to any one of embodiments 80-86, wherein cevostamab is administered at a frequency of Q4W for one or more cycles.
88. The method according to any one of embodiments 78 and 81-87, the use according to any one of embodiments 79 and 81-87, or the XmAb24306 and cevostamab according to any one of embodiments 80-87, wherein XmAb24306 is administered intravenously at a frequency of Q4W for at least seven four-week cycles, and wherein a first priming dose of 0.3 mg of cevostamab is administered intravenously on pre-phase Day 1, a second priming dose of 3.3 mg of cevostamab is administered intravenously between pre-phase Days 2-4, wherein the pre-phase is seven days, and, thereafter, at a dose of 132 mg of cevostamab is administered intravenously on Days 1 and 15 for six four-week cycles and on Day 1 for the seventh and any subsequent four-week cycles.
89. A method of treating multiple myeloma in a subject in need thereof, the method comprising administering to the subject a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30, wherein XmAb24306 is administered intravenously at a dose from about 0.02 mg/kg to about 0.06 mg/kg at a frequency of Q4W for at least seven four-week cycles, and wherein a first priming dose of 0.3 mg of cevostamab is administered intravenously on pre-phase Day 1, a second priming dose of 3.3 mg of cevostamab is administered intravenously between pre-phase Days 2-4, wherein the pre-phase is seven days, and, thereafter, at a dose of 132 mg of cevostamab is administered intravenously on Days 1 and 15 for six four-week cycles and on Day 1 for the seventh and any subsequent four-week cycles.
90. Use of a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30 in the manufacture of one or more medicaments for treating multiple myeloma in a subject in need thereof, wherein XmAb24306 is formulated to be administered intravenously at a dose from about 0.02 mg/kg to about 0.06 mg/kg at a frequency of Q4W for at least seven four-week cycles, wherein cevostamab is formulated to be administered intravenously at a dose of 132 mg on Days 1 and 15 for six four-week cycles and on Day 1 for the seventh and any subsequent four-week cycles; and wherein the treatment further comprises a seven-day pre-phase in which a first priming dose of 0.3 mg of cevostamab is administered intravenously on pre-phase Day 1, and a second priming dose of 3.3 mg of cevostamab is administered intravenously between pre-phase Days 2-4.
91. A therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30 for use in treating multiple myeloma in a subject in need thereof, wherein XmAb24306 is administered intravenously at a dose from about 0.02 mg/kg to about 0.06 mg/kg at a frequency of Q4W for at least seven four-week cycles, and wherein a first priming dose of 0.3 mg of cevostamab is administered intravenously on pre-phase Day 1, a second priming dose of 3.3 mg of cevostamab is administered intravenously between pre-phase Days 2-4, wherein the pre-phase is seven days, and, thereafter, at a dose of 132 mg of cevostamab is administered intravenously on Days 1 and 15 for six four-week cycles and on Day 1 for the seventh and any subsequent four-week cycles.

### EXAMPLES

### Example 1: In Vitro Pharmacologic Activity of Cevostamab Combination Therapy with XmAb24306

Frozen vials of bone marrow mononuclear cells (BMMC) from multiple myeloma subjects were purchased from Discovery Life Sciences. The samples were thawed following the instructions from the manufacturer. The samples were cultured in RPMI with 10% fetal calf serum (FCS) with 100 ng/mL recombinant human IL-6 overnight. The live BMMC were isolated by Ficoll density-gradient centrifugation. 3×10⁵ viable cells were pretreated with 10 mg/ml XmAb24306 for 24 hours before 15 ng/ml cevostamab was added for combination treatment, or medium as single agent treatment in 96-well plates. The treatments were continued for an additional 72 hours. At the end of the treatment, the cells were collected, and the myeloma cell lysis was quantified by flow cytometric analysis. The quantification of absolute cell counts was determined by using the CountBright™ Absolute Counting Beads (ThermoFisher Scientific, #C36950). The multiple myeloma tumor cells were defined as CD38^{high}CD138⁺. The killing activity was calculated as: {(number of live target cells without treatment−number of live target cells with treatment)/(number of live target cells without treatment)}×100%.

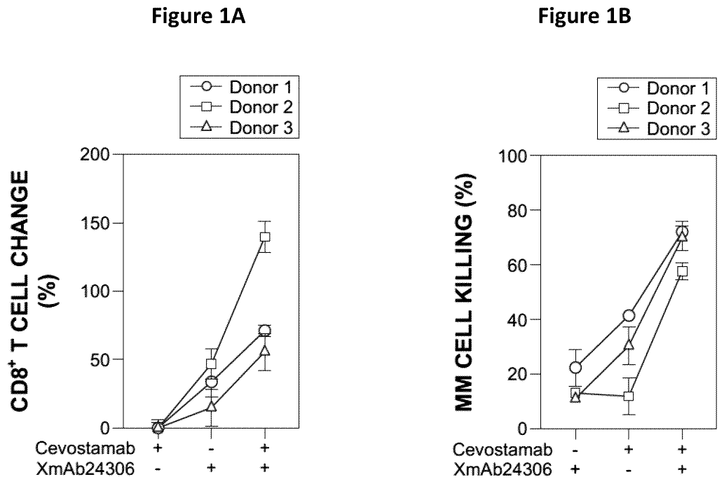

In vitro pharmacologic activity of cevostamab combination with XmAb24306 was evaluated. Primary BMMC from multiple myeloma (MM) subjects (n=3) with ratio of autologous CD8⁺ T cells:tumor cells varied from 0.1:1 to 2:1 were treated. The effect of treatment with either XmAb24306 alone or cevostamab alone was compared to the effect of treatment with the combination of XmAb24306a and cevostamab. Cevostamab alone, at 15 ng/mL, showed 10%-40% tumor cell lysis (FIG. 1B) without changing CD8⁺ T cell number (FIG. 1A). Treatment with 10 mg/mL XmAb24306 slightly augmented CD8⁺ T cell numbers (FIG. 1A) with minimal tumor cell lysis (FIG. 1B). Co-treatment with both XmAb24306 and cevostamab synergistically increased the CD8⁺ T cell numbers (FIG. 1A) and resulted in synergistic enhancement of cevostamab-mediated target cell killing (FIG. 1B).

Human peripheral blood mononuclear cells (PBMC) were isolated from whole blood of healthy donors by Ficoll gradient. A co-culture of PBMCs with a multiple myeloma cell line (e.g., MOLP-2 cells) was prepared in a 3:1 ratio in 96 well plates in the presence or absence of 5000 pM of cevostamab for 30 minutes, 2 hours, 4 hours, 24 hours and 48 hours. In the end of the incubation, cells were collected and stained with surface markers as following: CD3-BV421, CD4-APC-Cy7, CD8-BUV395, CD56-APC, CD122-PE, CD25-FITC and CD69-PE-Cy7. The expression level of CD69, CD25 and CD122 on CD4⁺ T cells, CD8⁺ T cells and NK cells were analyzed by flow cytometry.

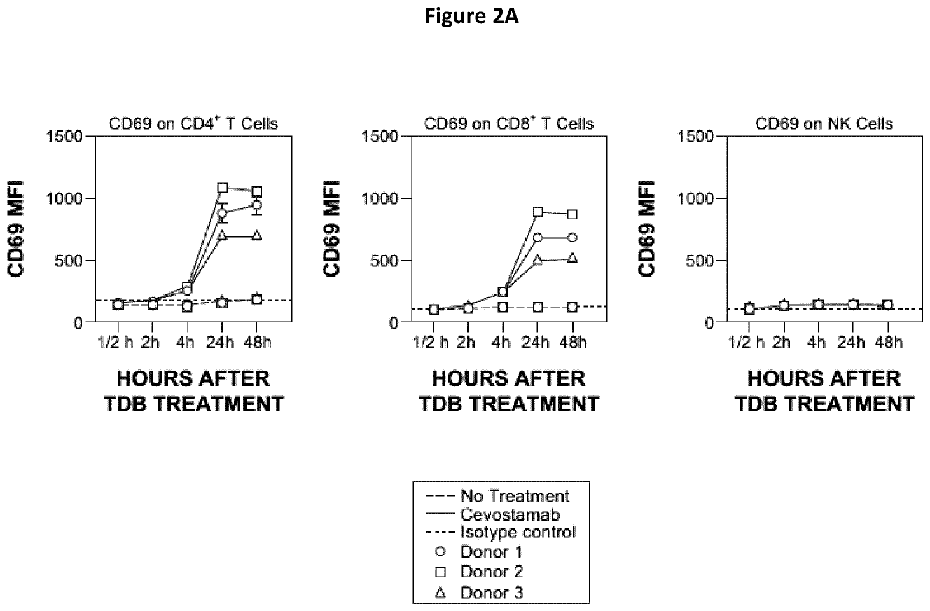

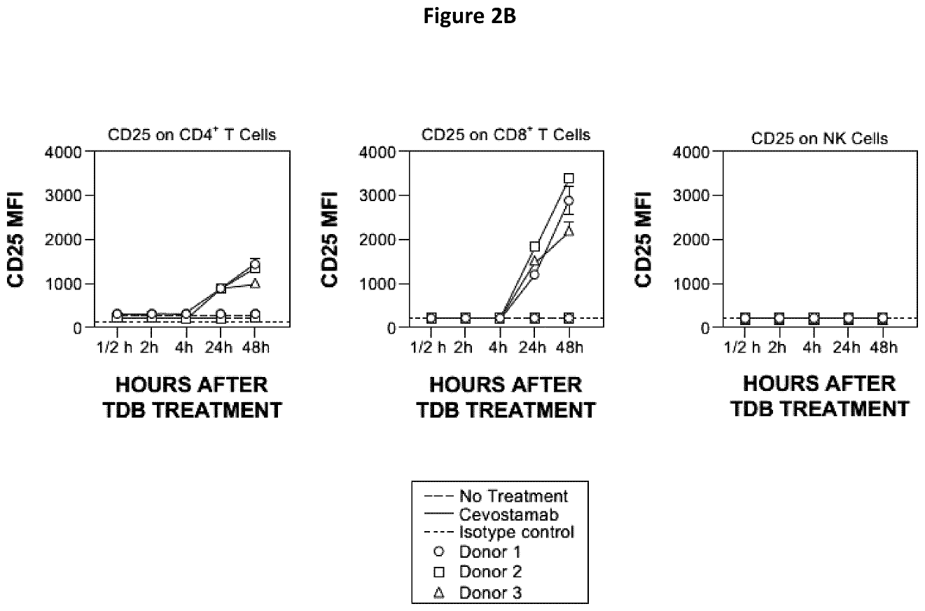

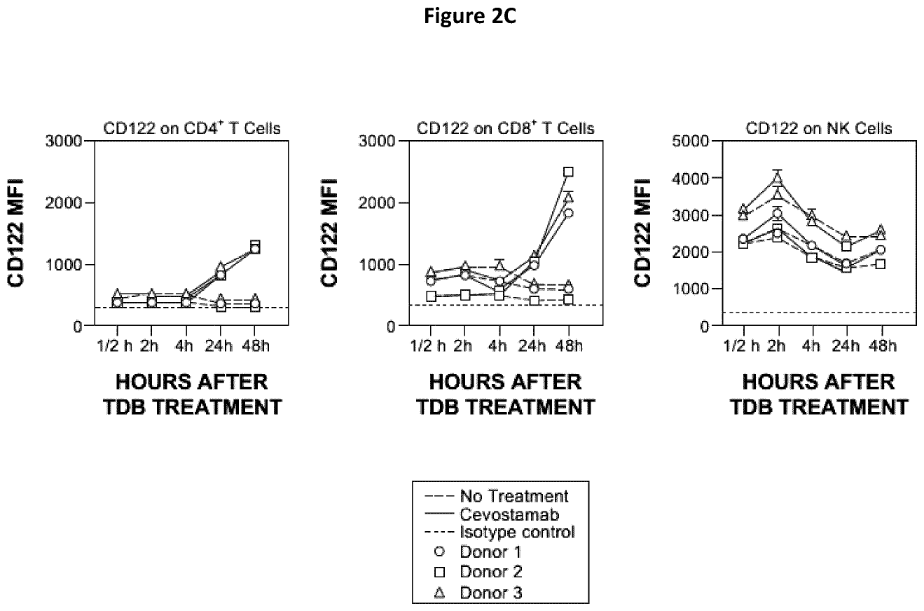

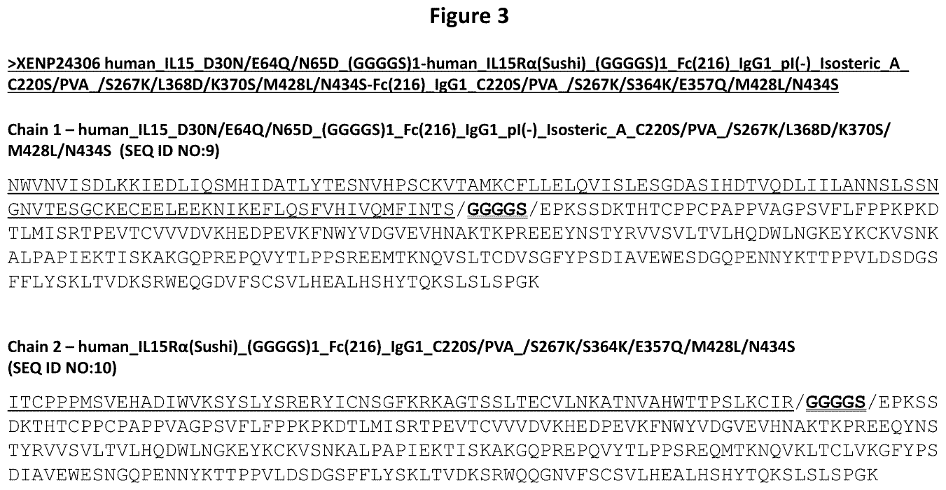

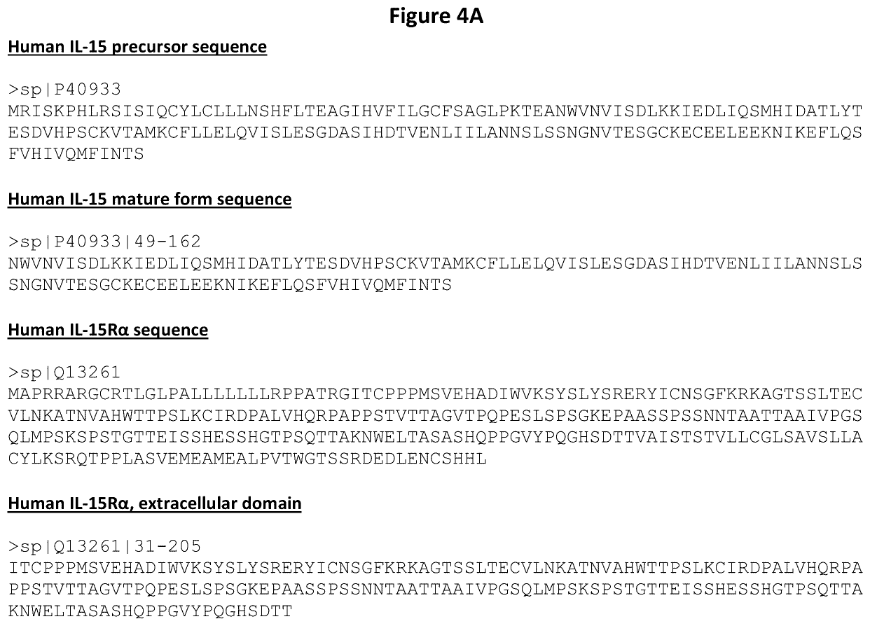

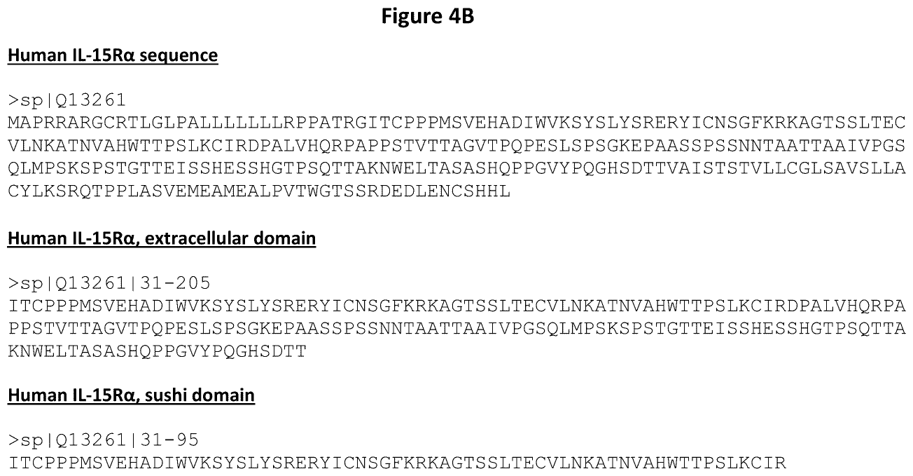

CD69, CD25 and CD122 expression on T cell and natural killer (NK) cells upon cevostamab treatment was assessed by co-culturing human PBMC and MOLP-2 cells in presence or absence of cevostamab (FIGS. 2A-2C). MOLP-2 was identified as a benchmark cell line expressing similar levels of FcRH5 as plasma cells and MM tumor cells (Li et al. 2017). CD69 (an early T cell activation marker) expression was detectable on both CD4⁺ T cells and CD8⁺ T cells at 4 hour cevostamab treatment and reached the peak level in 24 hours (FIG. 2A). CD25 expression level increased after 24 hour treatment in both CD4⁺ T cells and CD8⁺ T cells (FIG. 2B). No significant change of CD69 or CD25 was observed in NK cells after treatment (FIGS. 2A and 2B). CD122 was expressed at low levels in unstimulated peripheral blood CD4⁺ T cells and CD8⁺ T cells. Stimulation of T cells with 5000 pM cevostamab induced the expression of CD122 at 24-48h on CD4⁺ T cells and CD8⁺ T cells, but had little effect on NK cells, which express high levels of CD122 at the baseline (FIG. 2C).

### Example 2: Combination Therapy, Open-Label, Multicenter, Global, Dose-Escalation Study of XmAb24306 in Combination with Cevostamab

A combination therapy, open-label, multicenter, global, dose-escalation study to evaluate the safety, tolerability, pharmacokinetics and activity of the FcRH5xCD3 bispecific antibody cevostamab alone or in combination with XmAb24306 will be conducted in subjects with blood cancer (e.g., relapsed or refractory multiple myeloma (R/R MM)) who have received a minimum of three prior lines of treatment, including at least one immunomodulatory drug (IMiD), one proteasome inhibitor (PI), and one anti-CD38 monoclonal antibody.

The study consists of a screening period of up to 28 days, a treatment period (up to 1 year or longer), and a minimum follow-up period of 90 days after treatment.

Subjects in the combination study arm will be enrolled in two stages: a dose-escalation stage and an expansion stage.

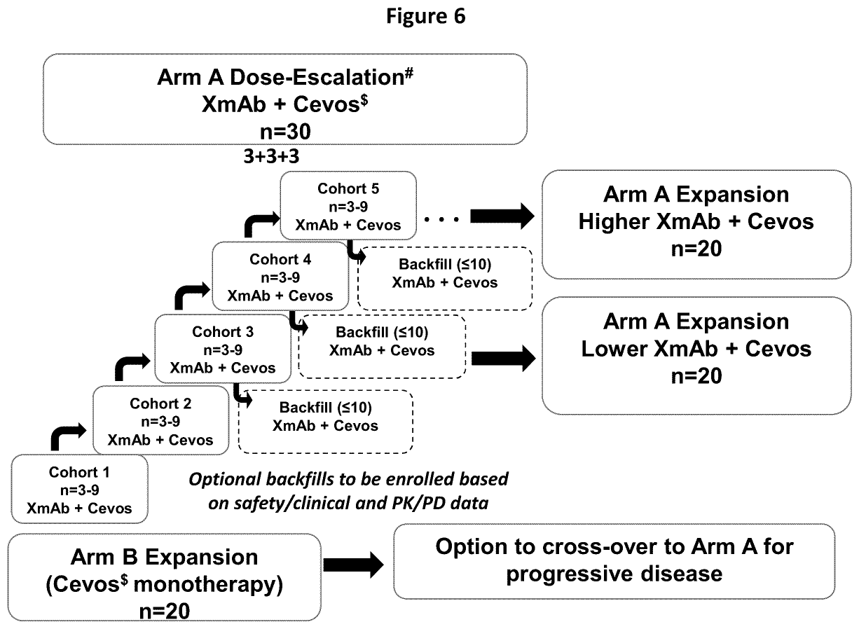

Cohorts of 3 to 9 subjects with a blood cancer (e.g., R/R MM) will be enrolled in the dose-escalation stage for the combination therapy portion of the study. XmAb24306 at escalating doses will be administered by IV infusion and 132 mg cevostamab will be administered intravenously following a 3+3+3 design (FIG. 6) to determine the maximum tolerated dose (MTD) or maximum administered dose (MAD) for XmAb24306 in combination with cevostamab.

Cevostamab will be initially administered using a split-dose priming schedule. Two cevostamab priming doses will be administered during a pre-phase cycle with the first priming dose (0.3 mg) administered on Day 1 and the second priming dose (3.3 mg) administered on Day 2, 3 or 4 pending resolution of infusion-related events including cytokine release syndrome (CRS), and with a minimum interval of 20 hours from the end of infusion of the first cevostamab priming dose to the initiation of second priming dose. Thereafter, the cevostamab target dose (132 mg) will be administered every 2 weeks (on Day 1 and Day 15) in Cycles 1-6 and every 4 weeks (on Day 1) in Cycles 7 and beyond (see FIG. 5).

The starting dose for XmAb24306 will be 0.02 mg/kg administered IV. The first dose of XmAb24306 will be administered at least 24 hours after the first target dose of cevostamab on Cycle 1 Days 2-4 once the subject has cleared safety parameters. Thereafter, XmAb24306 will be administered every 4 weeks on Day 1 of each cycle in Cycle 2 and beyond (see FIG. 5). In dose-escalation, the DLT assessment window is defined as Day 1 to Day 28 of Cycle 1.

The cevostamab monotherapy arm will enroll approximately 20 subjects and provide contemporaneous comparison data for the combination therapy described above. In the event that enrollment into the combination therapy arm will be restricted to specific populations of interest, the same restriction may be applied to the enrollment in the monotherapy arm.

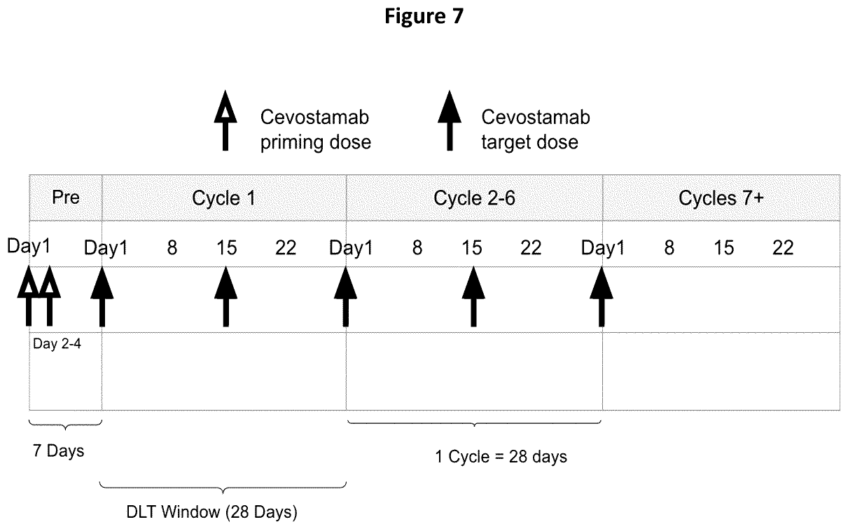

The cevostamab dosing schedule in the monotherapy arm will be a split-dose priming schedule with two escalating cevostamab priming doses that will be administered during the pre-phase period with the first priming dose of 0.3 mg to be administered on Day 1 and the second priming dose of 3.3 mg to be administered between Days 2 to 4, and target doses of 132 mg will be administered every 2 weeks (on Day 1 and Day 15) in Cycles 1 to 6 and every 4 weeks (on Day 1) in Cycle 7 and beyond (see, FIG. 7).

Subjects who are initially treated with the cevostamab monotherapy who have been on treatment for at least 1 cycle with a disease response assessment of stable disease (SD) or better but who subsequently progress while on study treatment may benefit from treatment with XmAb24306 in combination with cevostamab. Accordingly, such subjects may be eligible to crossover into the combination arm of the trial. Without being bound by theory, the rationale for this crossover design is that the addition of XmAb24306 to cevostamab may increase the proliferation, survival and/or cytotoxicity of CD8⁺ T cells resulting in enhanced antitumor activity as compared with single-agent cevostamab. Subjects who crossover from cevostamab monotherapy to XmAb24306 and cevostamab combination therapy may receive up to 12 months of combination therapy, or longer.

Subjects that are treated with cevostamab as a single agent who develop progressive disease and/or who are no longer deriving clinical benefit may be eligible to receive treatment with XmAb24306 in combination with cevostamab provided that the following criteria are met:
Subject must have been on treatment in the cevostamab monotherapy arm for at least 1 cycle with a disease response assessment of stable disease (SD) or better before progressing while on study treatment;
Treatment with XmAb24306 and cevostamab after progression on single-agent XmAb24306 must be considered acceptable as determined after a careful assessment and discussion of the benefit-risk balance;
Subjects must meet all the inclusion and exclusion criteria;
Patients must have documented disease progression by IMWG Uniform Response Criteria;
Subjects must not have experienced toxicity during the final dose of cevostamab monotherapy study treatment that would preclude treatment with the combination of XmAb24306 and cevostamab;
Any cevostamab-related adverse events must have resolved to Grade≤1 or to baseline grade and/or meet protocol-specified criteria for dosing for specific adverse events on or before the first day of treatment in the combination study (i.e., Example 2). Exceptions may be allowed.

The subjects who will qualify to cross over, the time between the final dose of cevostamab monotherapy administered and the planned first dose of cevostamab to be administered in the combination therapy with XmAb24306 (i.e., Example 2) must be ≥2 weeks. Depending on the length of this interval, the subject will also repeat the Pre-Phase prior to cycle 1 as follows:
If the interval between the final dose of cevostamab monotherapy and the planned first dose of cevostamab in the combination therapy with XmAb24306 is ≥2 weeks but <6 weeks, subjects will restart treatment on Cycle 1 Day 1 for the combination therapy, and cevostamab target dose will be administered on Day 1 and XmAb24306 administered between Days 2 to 4.
If the interval between the final dose of cevostamab monotherapy and the initial dose of cevostamab in the combination therapy with XmAb24306 is 8 weeks or longer, repeating Pre-Phase dosing will be mandatory.

If the interval between the final dose of cevostamab monotherapy arm and the initial dose of cevostamab in the combination therapy with XmAb24306 is at least 6 weeks but <8 weeks, it will be determined if repeat Pre-Phase dosing is required prior to Cycle 1.

All subjects in both arms of the trial will be closely monitored for adverse events throughout the study and for at least 90 days after the last dose of the study treatment. All adverse events including serious adverse events and adverse events of special interest will be reported until 90 days after the final dose of study treatment or until initiation of new systemic anti-cancer therapy, whichever occurs first. Adverse events will be graded according to the National Cancer Institute Common Terminology Criteria for Adverse Events, Version 5.0 (NCI CTCAE v5.0) with the exception of CRS, which will be graded according to American Society of Transplantation and Cellular Therapy (ASTCT) Consensus Grading for Cytokine Release Syndrome. Tocilizumab will be administered as appropriate for treatment-emergent CRS. Tocilizumab will be administered at a dose of 8 mg/kg IV (8 mg/kg for patients ≥30 kg weight only; 12 mg/kg for patients <30 kg weight; doses not to exceed 800 mg per infusion) and repeated every 8 hours as necessary (up to a maximum of four doses).

Subjects in both arms of the trial will undergo disease assessments at screening (baseline) and at regular intervals during the study, which will be evaluated according to International Myeloma Working Group [IMWG] Uniform Response Criteria. Subjects with acceptable toxicity and evidence of clinical benefit may continue to receive treatment with XmAb24306 and cevostamab for up to 12 months or until disease progression (as determined according to IMWG criteria) or unacceptable toxicity, whichever occurs first. After completion of 12 months of study treatment, patients who have not experienced disease progression may continue study treatment if they agree to a bone marrow aspirate/biopsy and their overall IMWG disease assessment does not indicate a stringent complete response (sCR). If bone marrow assessment is not performed, or if bone marrow demonstrates sCR, patient will no longer be able to continue study treatment. Patients who initially respond to study treatment, but subsequently develop recurrent or progressive disease either after the completion of therapy or after a dose delay of more than 28 days (i.e., no study treatment >42-56 days depending on when the delay occurs during study treatment), may be eligible for re-treatment.

Subjects who complete study treatment will continue to undergo tumor and additional assessments until disease progression, start of new anti-cancer therapy, or withdrawal from study participation, whichever occurs first.

Subjects who permanently discontinue XmAb24306 and cevostamab will return to the clinic for a treatment discontinuation visit within 30 days after the final dose of study treatment or the initiation of another systemic anti-cancer therapy, whichever occurs first. The visit at which response assessment shows progressive disease may be used as the treatment discontinuation visit.

To characterize the pharmacokinetics, immunogenicity response, and PD properties of XmAb24306 and/or cevostamab, blood samples will be taken at various timepoints before and after dosing.

The safety objective for this study is to evaluate the safety and tolerability of the combination of XmAb24306 and cevostamab on the basis of the following endpoints:
Incidence and severity of adverse events, with severity determined according to NCI CTCAE v5.0 and ASTCT Consensus Grading for Cytokine Release Syndrome;
Change from baseline in targeted vital signs;
Change from baseline in targeted clinical laboratory test results; and
Change from baseline in ECG parameters.

The pharmacokinetic (PK) objective for this study is to characterize the PK profile of XmAb24306 in combination with cevostamab on the basis of the following endpoints:
Serum concentration of cevostamab at specified timepoints;
Serum concentration of XmAb24306 at specified timepoints;
PK parameters of cevostamab; and
PK parameters of XmAb24306.

The exploratory PK objectives for this study are as follows:
To evaluate potential relationships between serum concentration or PK parameters for XmAb24306 and/or cevostamab and PD biomarkers, including but not limited to cytokine release, T-cell number, and T-cell activation state; and
To evaluate potential relationships between selected covariates and serum concentration of PK parameters for XmAb24306 and/or cevostamab to study treatment.

The activity objective for this study is to make a preliminary assessment of the activity of XmAb24306 when administered in combination with cevostamab, on the basis of the following endpoints:
Objective response rate (ORR), defined as the proportion of patients with a best overall response of sCR, complete response (CR), very good partial response (VGPR), or partial response (PR), as determined by the investigator according to International Myeloma Working Group (IMWG) criteria
Rate of CR/sCR, defined as the proportion of patients achieving a CR or sCR as determined by the investigator on two consecutive occasions
Rate of VGPR or better, defined as the proportion of patients achieving a VGPR or better as determined by the investigator on two consecutive occasions

The exploratory activity objective for this study is to make a preliminary assessment of the activity of XmAb24306 when administered in combination with cevostamab, on the basis of the following endpoints:
Duration of response (DOR), defined as the time from the first occurrence of a documented confirmed objective response (sCR, CR, VGPR, or PR) to disease progression or death from any cause during the study (defined as within 30 days after the final dose of study drug), as determined by the investigator according to IMWG criteria
Progression-free survival (PFS), defined as the time from the first study treatment to the first occurrence of disease progression or death from any cause during the study (defined as within 30 days after the final dose of study drug), whichever occurs first, as determined by the investigator according to IMWG criteria
Time to first response (for patients who achieve a response of PR or better), defined as the time from initiation of study treatment to achieving a confirmed PR or better
Time to best response (for patients who achieve a response of PR or better)
Rate of minimal residual disease (MRD) negativity (for patients who achieve a response of CR or sCR), defined as the proportion of patients who achieve MRD negativity as determined using next-generation sequencing (NGS) on bone marrow aspirate
Overall survival (OS), defined as the time from initiation of study treatment to death from any cause

For subjects treated with tocilizumab, an additional exploratory activity objective for this study is to make a preliminary assessment of the efficacy of tocilizumab in ameliorating the symptoms of cytokine release syndrome (CRS) following administration of study treatment on the basis of the following endpoint:
Outcome of CRS following the administration of tocilizumab.

The immunogenicity objective for this study is to evaluate the immune response to XmAb24306 in combination with cevostamab on the basis of the following endpoints:
Prevalence of anti-drug antibodies (ADAs) against cevostamab at baseline and incidence of ADAs against cevostamab during the study
Prevalence of anti-drug antibodies (ADAs) against XmAb24306 at baseline and incidence of ADAs against XmAb24306 during the study.

The exploratory immunogenicity objective for this study is to evaluate potential effects of ADAs on the basis of the following endpoints:
between status of ADAs against cevostamab and safety, PK, or activity endpoints
Relationship between status of ADAs against XmAb24306 and safety, PK, or activity endpoints.

The exploratory biomarker objectives for this study are the following:
To evaluate the activity of the study treatment in driving MRD negativity in patients with R/R MM, on the basis of the following endpoints:
   MRD negativity at time of first CR; and
   MRD negativity after 12 months of study treatment.
To identify and/or evaluate biomarkers that are predictive of response to XmAb24306 and cevostamab (i.e., predictive biomarkers), are early surrogates of activity, are associated with progression to a more severe disease state (i.e., prognostic biomarkers), are associated with acquired resistance to XmAb24306 and/or cevostamab, are associated with susceptibility to developing adverse events or can lead to improved adverse event monitoring or investigation (i.e., safety biomarkers), can provide evidence of XmAb24306 and/or cevostamab activity (i.e., pharmacodynamic (PD) biomarkers), or can increase the knowledge and understanding of disease biology and drug safety, on the basis of the following endpoint:
   Relationship between biomarkers in blood and bone marrow aspirate/biopsy and safety, PK, activity, immunogenicity, or other biomarker endpoints.

1. A method of treating a blood cancer in a subject in need thereof, the method comprising administering to the subject a therapeutically effective amount of (a) an IL15-IL15Rα heterodimeric Fc-fusion protein and (b) an FcRH5xCD3 bispecific antibody or a fragment thereof that binds both antigens.

2.-3. (canceled)

4. The method according to claim 1, wherein the IL15-IL15Rα heterodimeric Fc-fusion protein comprises: (a) a first fusion protein comprising an interleukin-15 (IL-15) protein covalently attached to the N-terminus of a first Fc domain via a first domain linker, wherein the IL-15 protein comprises the amino acid sequence of SEQ ID NO: 5 and wherein the first Fc domain is a variant of a human IgG1 Fc domain; and (b) a second fusion protein comprising an IL-15 receptor alpha (IL-15Rα) protein fragment covalently attached to the N-terminus of a second Fc domain via a second domain linker, wherein the IL-15Rα protein comprises the amino acid sequence of SEQ ID NO: 4 and wherein the second Fc domain is a variant of a human IgG1 Fc domain.

5. The method according to claim 4, wherein the IL15-IL15Rα heterodimeric Fc-fusion protein comprises: (a) a first fusion protein comprising an interleukin-15 (IL-15) protein covalently attached to the N-terminus of a first Fc domain via a first domain linker, wherein the IL-15 protein comprises the amino acid sequence of SEQ ID NO: 5 and wherein the first Fc domain comprises the amino acid sequence of SEQ ID NO: 6; and (b) a second fusion protein comprising an IL-15 receptor alpha (IL-15Rα) protein fragment covalently attached to the N-terminus of a second Fc domain via a second domain linker, wherein the IL-15Rα protein comprises the amino acid sequence of SEQ ID NO: 4 and wherein the second Fc domain comprises the amino acid sequence of SEQ ID NO: 7.

6.-10. (canceled)

11. The method according to claim 4, wherein the heterodimeric protein comprises a first fusion protein comprising the amino acid sequence set forth in SEQ ID NO: 9, and a second fusion protein comprising the amino acid sequence set forth in SEQ ID NO: 10.

12. The method according to claim 4, wherein the IL15-IL15Rα heterodimeric Fc-fusion protein is XmAb24306.

13. The method according to claim 1, wherein the FcRH5xCD3 bispecific antibody or a fragment thereof that binds both antigens comprises an anti-FcRH5 light chain variable region, an anti-FcRH5 heavy chain variable region, an anti-CD3 light chain variable region, and an anti-CD3 heavy chain variable region.

14. The method according to claim 13, wherein the anti-FcRH5 light chain variable region comprises a light chain complementarity determining region-1 (CDR-L1) comprising the amino acid sequence of SEQ ID NO: 11, a CDR-L2 comprising the amino acid sequence of SEQ ID NO: 12, and a CDR-L3 comprising the amino acid sequence of SEQ ID NO: 13; and the anti-FcRH5 heavy chain variable region comprises a heavy chain complementarity determining region-1 (CDR-H1) comprising the amino acid sequence of SEQ ID NO: 14, a CDR-H2 comprising the amino acid sequence of SEQ ID NO: 15, and a CDR-H3 comprising the amino acid sequence of SEQ ID NO: 16.

15.-18. (canceled)

19. The method according to claim 13, wherein the anti-CD3 light chain variable region comprises a light chain complementarity determining region-1 (CDR-L1) comprising the amino acid sequence of SEQ ID NO: 21, a CDR-L2 comprising the amino acid sequence of SEQ ID NO: 22, and a CDR-L3 comprising the amino acid sequence of SEQ ID NO: 23; and the anti-CD3 heavy chain variable region comprises a heavy chain complementarity determining region-1 (CDR-H1) comprising the amino acid sequence of SEQ ID NO: 24, a CDR-H2 comprising the amino acid sequence of SEQ ID NO: 25, and a CDR-H3 comprising the amino acid sequence of SEQ ID NO: 26.

20.-23. (canceled)

24. The method according to claim 13, wherein the anti-FcRH5 light chain variable region comprises the amino acid sequence of SEQ ID NO: 17; the anti-FcRH5 heavy chain variable region comprises the amino acid sequence of SEQ ID NO: 18; the anti-CD3 light chain variable region comprises the amino acid sequence of SEQ ID NO: 27; and the anti-CD3 heavy chain variable region comprises the amino acid sequence of SEQ ID NO: 28.

25. The method according to claim 13, wherein the anti-FcRH5 light chain comprises the amino acid sequence of SEQ ID NO: 19; the anti-FcRH5 heavy chain comprises the amino acid sequence of SEQ ID NO: 20; the anti-CD3 light chain comprises the amino acid sequence of SEQ ID NO: 29; and the anti-CD3 heavy chain comprises the amino acid sequence of SEQ ID NO: 30.

26. The method according to claim 13, wherein the FcRH5xCD3 bispecific antibody is cevostamab.

27. The method according to claim 1, wherein said blood cancer is selected from the group consisting of leukemia, acute myeloid leukemia, adult acute lymphoblastic leukemia, chronic lymphocytic leukemia, lymphoma, non-Hodgkin's lymphoma, B-cell non-Hodgkin's lymphoma, and multiple myeloma.

28.-34. (canceled)

35. The method according to claim 1, wherein the IL15-IL15Rα heterodimeric Fc-fusion protein is administered at a dose selected from the group consisting of about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.1 mg/kg, about 0.12 mg/kg, about 0.16 mg/kg, about 0.2 mg/kg, about 0.24 mg/kg and about 0.32 mg/kg body weight.

36.-38. (canceled)

39. The method according to claim 1, wherein the IL15-IL15Rα heterodimeric Fc-fusion protein is administered at a frequency selected from the group consisting of Q1W, Q2W, Q3W, Q4W, Q5W and Q6W.

40.-45. (canceled)

46. The method according to claim 1, wherein said FcRH5xCD3 bispecific antibody or fragment thereof that binds both antigens is administered at a frequency selected from the group consisting of Q1W, Q2W, Q3W, Q4W, Q5W and Q6W in one or more cycles.

47.-52. (canceled)

53. The method according to claim 1, wherein the FcRH5xCD3 bispecific antibody is administered at a dose of about 132 mg to about 198 mg.

54.-57. (canceled)

58. The method, wherein one or more priming doses of the FcRH5xCD3 bispecific antibody or a fragment thereof that binds both antigens is administered to the subject during a pre-phase before the first treatment cycle.

59.-68. (canceled)

69. The method according to claim 1, wherein the method further comprises administering to the subject a therapeutically effective amount of tocilizumab.

70.-77. (canceled)

78. A method of treating multiple myeloma in a subject in need thereof, the method comprising administering to the subject a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30, wherein XmAb24306 is administered intravenously at a dose from about 0.02 mg/kg to about 0.06 mg/kg and cevostamab is administered intravenously at a dose of about 132 mg.

79.-88. (canceled)

89. A method of treating multiple myeloma in a subject in need thereof, the method comprising administering to the subject a therapeutically effective amount of (a) XmAb24306, wherein XmAb24306 comprises a first monomer comprising the amino acid sequence of SEQ ID NO: 9 and a second monomer comprising the amino acid sequence of SEQ ID NO: 10, and (b) cevostamab, wherein cevostamab comprises an anti-FcRH5 light chain comprising the amino acid sequence of SEQ ID NO: 19, an anti-FcRH5 heavy chain comprising the amino acid sequence of SEQ ID NO: 20, an anti-CD3 light chain comprising the amino acid sequence of SEQ ID NO: 29, and an anti-CD3 heavy chain comprising the amino acid sequence of SEQ ID NO: 30, wherein XmAb24306 is administered intravenously at a dose from about 0.02 mg/kg to about 0.06 mg/kg at a frequency of Q4W for at least seven four-week cycles, and wherein a first priming dose of 0.3 mg of cevostamab is administered intravenously on pre-phase Day 1, a second priming dose of 3.3 mg of cevostamab is administered intravenously between pre-phase Days 2-4, wherein the pre-phase is seven days, and, thereafter, at a dose of 132 mg of cevostamab is administered intravenously on Days 1 and 15 for six four-week cycles and on Day 1 for the seventh and any subsequent four-week cycles.

90.-91. (canceled)


SEQUENCE LISTING (31 sequences)

SEQ ID NO: 1 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 4 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 5 - protein, 114 aa, synthetic construct
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 6 - protein, 231 aa, Homo sapiens
EPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 7 - protein, 231 aa, Homo sapiens
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 8 - protein, 5 aa, Homo sapiens
GGGGS

SEQ ID NO: 9 - protein, 350 aa, Homo sapiens
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 10 - protein, 301 aa, Homo sapiens
ITCPPPMSVEHADIWVKSYSLYSRERYICNSGFKRKAGTSSLTECVLNKATNVAHWTTPSLKCIRGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 11 - protein, 11 aa, synthetic construct
KASQDVRNLVV

SEQ ID NO: 12 - protein, 7 aa, synthetic construct
SGSYRYS

SEQ ID NO: 13 - protein, 9 aa, synthetic construct
QQHYSPPYT

SEQ ID NO: 14 - protein, 5 aa, synthetic construct
RFGVH

SEQ ID NO: 15 - protein, 16 aa, synthetic construct
VIWRGGSTDYNAAFVS

SEQ ID NO: 16 - protein, 12 aa, synthetic construct
HYYGSSDYALDN

SEQ ID NO: 17 - protein, 107 aa, synthetic construct
DIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIK

SEQ ID NO: 18 - protein, 120 aa, synthetic construct
EVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSS

SEQ ID NO: 19 - protein, 214 aa, synthetic construct
DIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 450 aa, synthetic construct
EVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 17 aa, synthetic construct
KSSQSLLNSRTRKNYLA

SEQ ID NO: 22 - protein, 7 aa, synthetic construct
WTSTRKS

SEQ ID NO: 23 - protein, 12 aa, synthetic construct
HYYGSSDYALDN

SEQ ID NO: 24 - protein, 5 aa, synthetic construct
SYYIH

SEQ ID NO: 25 - protein, 17 aa, synthetic construct
WIYPENDNTKYNEKFKD

SEQ ID NO: 26 - protein, 10 aa, unidentified
DGYSRYYFDY

SEQ ID NO: 27 - protein, 112 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 28 - protein, 119 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 29 - protein, 219 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 449 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT